(19) United States
(12) Patent Application Publication
(10) Pub. No.: US 2026/0151116 A1
(43) Pub. Date: Jun. 4, 2026

(54) HORMONAL LEVELS DETECTION THROUGH VOICE BIOMARKERS FOR WOMEN'S BRAIN HEALTH

(71) Applicant: Imen Clark, Phoenix, AZ (US)

(72) Inventor: Imen Clark, Phoenix, AZ (US)

(21) Appl. No.: 19/409,718

(22) Filed: Dec. 4, 2025

Related U.S. Application Data
(60) Provisional application No. 63/727,841, filed on Dec. 4, 2024.

(51) Int. Cl. A61B 10/00; A61B 5/00; A61B 5/0205; A61B 5/021; A61B 5/024; A61B 5/145; A61B 5/16; G10L 25/63; G10L 25/66

(52) U.S. Cl. CPC A61B 10/0012; A61B 5/0205; A61B 5/021; A61B 5/02405; A61B 5/14542; A61B 5/165; A61B 5/4803; A61B 5/7271; A61B 5/742; G10L 25/63; G10L 25/66

(57) ABSTRACT

The invention provides a non-invasive system and method for detecting hormonal levels through voice biomarkers to support women's brain health. The system captures voice samples, analyzes acoustic and semantic features, and integrates behavioral and physiological data to identify hormonal phases. An AI engine processes these inputs to deliver personalized, science-backed recommendations for cognitive resilience, emotional balance, and mental clarity. The platform supports daily engagement through guided voice journaling and integrates with wearable devices and health applications. Applications include early detection of hormone-related brain conditions, personalized lifestyle interventions, and large-scale research into hormonal influences on brain health. Privacy-preserving architectures ensure secure data handling while enabling longitudinal tracking and clinical integration.

### CROSS-REFERENCE TO RELATED APPLICATION

This application claims the benefit of and priority to U.S. Provisional Patent Application No. 63/727,841, filed Dec. 4, 2024, the contents of which are incorporated herein by reference.

### FIELD OF THE INVENTION

This invention relates to a non-invasive system and method for detecting hormonal levels through voice biomarkers to support women's brain health.

### BACKGROUND OF INVENTION

The relationship between hormonal fluctuations and brain health remains underexplored, particularly in the context of women's health. Current research efforts disproportionately focus on gender-neutral approaches, with less than 30% of research addressing the unique, gender-specific needs of women. This disparity is evident in brain health, where hormonal changes play a significant role in the development and manifestation of certain conditions.

Women are disproportionately affected by various brain-related conditions. For example, 70% of Alzheimer's patients are women. Women are also twice as likely to experience depression and are 3 to 4 times more likely to suffer from migraines. These statistics underscore the critical need for research and interventions tailored to women's unique physiological and hormonal profiles.

Hormonal fluctuations during key life stages, such as the menopausal transition, profoundly impact brain function. Over 60% of women experience brain fog, memory lapses, and mental fatigue during menopause. The loss of estrogen during this period contributes to an increased risk of Alzheimer's disease in women. Despite this, current medical and technological solutions fail to adequately address these unique challenges.

Furthermore, lifestyle modifications have the potential to prevent 40% of these conditions. This highlights the importance of developing targeted interventions that consider hormonal influences on the brain. However, existing health monitoring technologies largely overlook the impact of hormonal cycles, particularly on cognitive and emotional well-being.

Advances in digital health solutions, particularly those incorporating artificial intelligence (AI), offer an opportunity to address this knowledge gap. By combining behavioral data with advanced analytics, it is possible to create personalized interventions that support brain health through life's transitions.

Voice biomarker analysis represents a promising frontier in this field. Through the examination of semantics and acoustics in voice data, it is possible to detect hormonal fluctuations and their associated effects on the brain. This approach offers a non-invasive and accessible means of assessing brain health, providing actionable insights for both individuals and healthcare providers.

The invention described herein leverages speech analysis, conversational AI, and behavioral data collection to create a comprehensive digital health solution. By focusing on the detection of hormonal fluctuations and their impact on brain health, this technology aims to empower women with the tools and knowledge needed to support cognitive resilience and emotional well-being.

### SUMMARY OF THE INVENTION

This invention introduces a novel digital health solution that utilizes voice biomarkers to detect hormonal levels and their impact on brain health. The system employs speech analysis, integrating both acoustics and semantics, to monitor hormonal fluctuations. By analyzing behavioral and health data, the technology provides personalized, science-backed recommendations for maintaining brain health.

The AI-powered engine processes data points such as exercise habits, sleep patterns, dietary intake, stress levels, and social interactions. Additionally, it monitors physiological metrics including hormonal cycles, oxygen saturation, blood pressure, heart rate variability, and cholesterol levels. These inputs enable precise detection of hormonal phases and their cognitive and emotional impacts.

This solution is particularly geared toward addressing the unique challenges faced by women during hormonal transitions, such as menopause. By identifying early signs of hormonal imbalance, the system helps lower the risks associated with brain conditions such as Alzheimer's, depression, and migraines. Through daily voice journaling and interactive engagement, users gain actionable insights to enhance mental clarity, emotional well-being, and cognitive resilience.

The invention bridges the gap between research and practice, democratizing knowledge about women's brain health. It empowers users with tailored interventions, contributing to the prevention and early detection of brain-related conditions.

The subject invention discloses a computer-implemented method for multimodal hormonal health assessment comprises initiating, by a digital health platform, a user session in which a user is prompted to provide a voice sample via a microphone-enabled device, detecting active speech and capturing raw audio waveforms while concurrently logging contextual metadata including timestamp, device type, ambient noise estimate, and device orientation, buffering the captured audio and applying signal-conditioning operations including noise suppression, band-limiting, amplitude normalization, and silence trimming, segmenting the conditioned audio into overlapping analytical frames and, for each frame, computing acoustic features including at least one of fundamental frequency, jitter, shimmer, formant trajectories, spectral centroid, spectral roll-off, harmonic-to-noise ratio, glottal-flow indicators, and Mel-frequency cepstral coefficients, aggregating frame-level features into session-level statistics with temporal smoothing, converting the conditioned audio into text using automatic speech recognition, computing semantic features from the text including sentiment, emotional tone, linguistic complexity, stress-related keywords, speech rate, pause density, and hesitation markers, retrieving behavioral and physiological data streams associated with the session, temporally aligning the acoustic, semantic, behavioral, and physiological features into a synchronized dataset, inputting the synchronized dataset to a trained machine-learning model, generating from the model a predicted hormonal phase and an associated confidence score, and outputting the predicted hormonal phase for display together with individualized health recommendations.

The subject invention further a discloses a system for multimodal voice-based hormonal assessment comprises a microphone-enabled user device configured to capture raw voice input and contextual metadata, a preprocessing engine configured to buffer the raw audio and apply noise suppression, amplitude normalization, silence trimming, and windowing to produce conditioned audio frames, an acoustic-feature-extraction module configured to compute per-frame voice features including pitch, jitter, shimmer, formant trajectories, spectral measures, glottal indicators, and cepstral coefficients and to aggregate these features into smoothed session-level descriptors, an automatic speech-recognition engine configured to produce a text transcript from the conditioned audio, a semantic-analysis module configured to compute sentiment scores, emotional valence, linguistic-complexity metrics, stress-related lexical markers, speech-rate metrics, pause statistics, and hesitation markers from the transcript, a data-integration module configured to retrieve behavioral and physiological data streams, align such streams with the session timing, and construct a unified multimodal feature representation, and an inference engine comprising at least one trained machine-learning model configured to receive the unified feature representation, estimate a hormonal phase and a confidence value, and supply an output interface with phase predictions and associated recommendations.

The subject invention also discloses a computer-implemented method for behavioral data capture and integration in a hormonal-monitoring platform comprises presenting to a user one or more prompts requesting reflections on daily routines, sleep experience, exercise activity, diet, stress level, social interactions, and emotional state, receiving user responses in spoken or textual form and recording each response together with metadata including timestamp and prompt context, processing each response to extract structured behavioral attributes including at least hours slept, perceived sleep quality, exercise type and intensity, dietary categories, subjective stress level, and reported fatigue, converting the extracted attributes into normalized metadata fields suitable for longitudinal analysis, associating each behavioral record with concurrently or recently collected physiological and voice-derived data based on timestamp alignment, aggregating behavioral records produced within and across days into a rolling behavioral profile that captures both short-term variability and long-term habits, detecting anomalous or incomplete records in the behavioral profile, imputing missing behavioral values based on historical patterns and user-specific baselines, and generating a time-aligned behavioral-feature sequence that can be directly incorporated into hormonal-phase prediction models.

The subject invention discloses a system for behavioral monitoring in a hormonal health platform comprises a user-interface module configured to deliver guided and unguided prompts about sleep, exercise, diet, stress, mood, and social context and to receive spoken or textual responses from a user, a behavioral-processing module configured to parse responses to identify and quantify key behavioral attributes including duration and quality of sleep, exercise patterns, dietary behaviors, stress ratings, and self-reported fatigue, a metadata manager configured to timestamp each processed response and associate it with session context, a behavioral-aggregation engine configured to combine multiple records into rolling daily and weekly profiles and to compute summary statistics and trends, a quality-control module configured to detect missing, inconsistent, or outlier behavioral entries and to perform imputation and normalization based on historical behavior for the user, and an integration module configured to synchronize the resulting behavioral-feature timelines with voice-derived and physiological features for use in a hormonal-state inference engine.

The subject invention further discloses a computer-implemented method for physiological-data acquisition and alignment in a hormonal-monitoring platform comprises establishing a secure communication channel between the platform and at least one wearable or physiological sensor, periodically polling the wearable or sensor for updates to physiological parameters including heart-rate variability, resting heart rate, oxygen saturation, respiratory rate, electrodermal activity, body temperature, activity levels, and menstrual-cycle events, receiving physiological records and performing an initial validation to ensure completeness, correct formatting, and plausible timestamps, applying artifact-removal algorithms to the validated data to discard measurements corrupted by motion or connection loss, smoothing remaining physiological time series using one or more filters to reduce noise and erratic fluctuations, detecting short-term deviations from baseline including drops in heart-rate variability, spikes in temperature, irregular heart rate, or unexpected cycle events, generating derived physiological features summarizing trends over selected intervals, temporally aligning the physiological features with voice and behavioral data by correcting for sampling-rate differences and latency, and producing a synchronized physiological-feature representation suitable for multimodal hormonal-phase analysis.

The subject invention also discloses a system for physiological integration in a digital hormonal health platform comprises a connectivity module configured to establish secure links with wearable devices and physiological sensors, a polling controller configured to request and receive data relating to heart-rate variability, heart rate, oxygen saturation, respiratory rate, electrodermal activity, temperature, activity levels, and menstrual-cycle events at predefined intervals or in response to triggers, a validation and artifact-processing module configured to inspect incoming data for completeness and plausibility and to remove or correct motion-induced spikes, dropped segments, and corrupted values, a feature-extraction module configured to compute smoothed physiological trends and deviation metrics, a synchronization module configured to align physiological features with voice and behavioral features on a common temporal axis, and an output interface configured to provide the aligned physiological-feature streams to a multimodal inference engine for hormonal state prediction.

The subject invention discloses a computer-implemented method for multimodal hormonal-phase inference comprises receiving, at an analysis engine, a unified feature vector or sequence comprising standardized voice metrics, linguistic markers, behavioral attributes, physiological indicators, and optionally cognitive-assessment metrics for a user over a defined time window, normalizing the unified feature representation with respect to user-specific baselines and population-level distributions, inputting the normalized representation into at least one trained machine-learning model selected from neural networks, transformer-based models, probabilistic classifiers, or hybrid interpretable architectures, allowing the model to process temporal patterns and cross-feature interactions to infer probabilities associated with a plurality of candidate hormonal phases, adjusting the probability distribution based on contextual constraints including known cycle length or menopausal status, computing a final hormonal-phase prediction by selecting the most probable phase, calculating a confidence score that reflects internal model consistency and the completeness of the input data, and storing the hormonal-phase prediction and confidence score in association with the user's longitudinal record.

The subject invention further discloses a digital health system for multimodal hormonal-phase inference comprises a data-normalization module configured to standardize acoustic, semantic, behavioral, physiological, and cognitive features relative to individual baselines and population statistics, a feature-construction module configured to assemble standardized features into unified vectors or sequences across definable time windows, an inference engine comprising at least one trained machine-learning model configured to accept the unified representation and to produce probabilistic estimates for multiple hormonal-phase categories, a context-adjustment component configured to modify the probabilistic estimates in light of user-specific constraints such as average cycle length or known menopausal status, a decision component configured to select a hormonal-phase output and compute an associated confidence measure, and a storage subsystem configured to log the phase predictions and confidence measures for longitudinal tracking and downstream recommendation generation.

The subject invention further discloses a computer-implemented method for generating personalized hormonal-phase-dependent health recommendations comprises receiving as input a predicted hormonal phase and one or more associated risk indicators derived from multimodal analysis, querying a knowledge base storing a catalog of candidate interventions indexed by hormonal phase, risk type, and user characteristics, filtering the candidate interventions based on user-specific preferences, lifestyle constraints, clinical contraindications, and historical adherence patterns, estimating an expected benefit score for each remaining intervention using predictive models that consider prior user outcomes and similarity to outcomes of comparable users, selecting a subset of interventions ranked according to the expected benefit scores, translating the selected interventions into concrete, user-facing guidance messages that specify actionable behaviors, delivering the guidance messages through one or more interfaces including mobile notifications, in-application views, or synthesized voice output, monitoring subsequent behavioral and physiological data to infer adherence to and impact of the delivered interventions, and updating internal models and future intervention rankings based on observed adherence and outcome effectiveness.

The subject invention also discloses a system for personalized hormonal-phase-guided intervention delivery comprises a phase-input interface configured to receive hormonal-phase predictions and risk indicators, an intervention knowledge base storing evidence-based lifestyle, cognitive, nutritional, and stress-management interventions indexed by hormonal state and user type, a personalization engine configured to filter interventions according to user preferences, constraints, contraindications, and adherence history, a ranking engine configured to assign expected benefit scores to candidate interventions using learned models of intervention effectiveness, a guidance-generation module configured to transform ranked interventions into specific, context-aware recommendation messages, a delivery subsystem configured to present the messages via mobile, web, or voice channels, and a feedback engine configured to observe user behavior and health metrics following recommendation delivery and to update the personalization and ranking engines accordingly.

The subject invention further discloses a computer-implemented method for adaptive engagement and baseline recalibration in a hormonal health platform comprises generating, at scheduled or context-triggered times, a prompt inviting the user to perform a voice or text check-in, receiving a response or detecting nonresponse and measuring engagement features including response latency, duration, level of detail, and affective tone, computing an engagement score based on the measured features and classifying the user's engagement state along a continuum, adjusting the length, complexity, and frequency of subsequent prompts according to the engagement state to minimize burden and maintain participation, continuously updating statistical models representing the user's typical acoustic ranges, emotional-expression patterns, sleep and activity rhythms, and physiological baselines as new multimodal data are collected, detecting significant deviations from these baselines that exceed dynamically defined thresholds, and triggering increased monitoring, more intensive support measures, or suggestions for clinical consultation when the detected deviations suggest emerging hormonal or cognitive risk.

The subject invention discloses a system for clinician-facing reporting and telemedicine integration in a hormonal-monitoring platform comprises an aggregation engine configured to collect hormonal-phase predictions, voice-biomarker trends, behavioral profiles, physiological metrics, and cognitive-assessment results over configurable time intervals, a summarization module configured to compute phase statistics, risk indicators, and correlation metrics between hormonal phases and other features, a visualization engine configured to generate clinician-readable dashboards including charts, tables, and highlighted risk periods, a security module configured to apply medical-grade encryption to all clinician-bound data and to verify clinician identity and authorization before transmission, an audit-log subsystem configured to record each data transmission event in an immutable log, and a feedback interface configured to accept clinician comments or treatment-plan adjustments and to incorporate those inputs into the platform's recommendation and monitoring logic for the user.

The subject invention further discloses a computer-implemented method for privacy-preserving hormonal-phase detection using federated learning comprises capturing a user's audio on a local device, performing on-device preprocessing and feature extraction to derive acoustic and optional semantic descriptors, deleting the raw audio buffer after feature extraction, executing a locally stored inference model on the extracted features to generate a hormonal-phase prediction and summary metrics, storing only the prediction and summary metrics in local memory, periodically computing model update gradients from local data for the purpose of improving the inference model, encrypting and transmitting the gradients, but not the underlying user data or raw features, to a central aggregation server, aggregating the gradients from multiple devices at the server to refine a global model, distributing updated model parameters back to participating devices, and updating the local inference model using the received parameters so that predictive performance improves over time without centralized storage of personal raw audio.

The subject invention also discloses a system for privacy-preserving distributed hormonal-state inference comprises a user device including a microphone, a feature-extraction engine configured to derive acoustic and semantic features from captured audio and to delete raw audio after feature derivation, a local inference model configured to generate hormonal-state predictions from the features without transmitting user-identifiable data off-device, a federated-learning client module configured to compute encrypted model updates from local data and send them to a federated-learning server, the federated-learning server configured to aggregate updates from multiple devices into a refined global model and to transmit updated parameters back to devices, and a model-update module on each device configured to incorporate the updated parameters into the local inference model so that system-wide performance improves while maintaining user privacy.

The subject invention further discloses a computer-implemented method for constructing a temporally synchronized multimodal dataset for hormonal analysis, which comprises receiving a plurality of input streams including acoustic feature sequences derived from a user's voice, semantic feature sequences derived from corresponding transcripts, behavioral records containing timestamped lifestyle attributes, and physiological records containing timestamped sensor measurements, assigning or standardizing timestamps for each element in each stream relative to a common reference clock, interpolating or resampling streams with differing sampling rates so that all streams can be evaluated over a harmonized time grid, resolving conflicts between overlapping records based on recency and data-quality criteria, imputing missing values using user-specific baselines and, optionally, population-level priors, normalizing feature magnitudes across streams so that no single modality dominates downstream learning, and outputting a synchronized multimodal dataset that preserves temporal relationships among acoustic, semantic, behavioral, and physiological states for input into a hormonal-phase inference model.

The subject invention also discloses a system for temporal alignment and fusion of multimodal health data in a hormonal-monitoring platform, which comprises a timestamp-normalization module configured to map voice-derived, behavioral, and physiological records to a shared temporal frame of reference, a resampling engine configured to up sample or down sample feature sequences from different sensors to a common temporal resolution, a conflict-resolution component configured to reconcile overlapping or inconsistent measurements using predetermined priority rules and quality scores, a missing-data handler configured to apply imputation algorithms based on historical user patterns and cross-user statistics, a feature-scaling unit configured to standardize or normalize feature ranges across modalities, and a fusion output interface configured to generate aligned multimodal feature tensors usable by machine-learning models for hormonal-phase prediction.

The subject invention further discloses a computer-implemented method for engagement scoring and interaction adaptation in a hormonal health platform, which comprises issuing a prompt to the user via a mobile, web, or voice interface requesting a check-in, recording whether and when the user responds, analyzing the response to derive engagement features including latency between prompt and response, response duration, depth of content, and expressed affective tone, combining the engagement features into a scalar or categorical engagement score using a learned or rules-based model, classifying the user's engagement state as at least high, moderate, or low engagement, adapting future prompts by adjusting at least one of frequency, complexity, modality, and length in accordance with the engagement state to reduce burden and maximize participation, and periodically recalculating engagement trajectories over time to inform higher-level personalization and monitoring strategies.

The subject invention also discloses a system for adaptive prompting and engagement management in a hormonal-monitoring environment, which comprises a prompt-scheduling module configured to generate time-based or context-triggered invitations for user check-ins, an engagement-feature extractor configured to compute latency, duration, content richness, and emotional-tone metrics from user responses, an engagement-classification engine configured to assign an engagement state from the extracted metrics, a prompt-adaptation controller configured to select future prompt templates and delivery channels based on the current and historical engagement states, and a data interface configured to store engagement metrics alongside hormonal-phase predictions for subsequent correlation analysis and model refinement.

The subject invention further discloses a computer-implemented method for de-identification and research aggregation of hormonal health data, which comprises receiving multimodal user records including voice-derived features, behavioral attributes, physiological indicators, and hormonal-phase predictions, replacing direct user identifiers with pseudonymous tokens generated from a secure mapping service, generalizing or coarsening potentially identifying metadata such as location or exact timestamps according to predefined privacy policies, applying noise injection or other differential-privacy mechanisms to selected feature values to reduce re-identification risk, grouping de-identified records into cohorts based on demographic bands, hormonal patterns, or health characteristics, computing aggregate statistics and trend metrics within each cohort, and storing the de-identified cohort-level data in a research repository accessible through controlled interfaces.

The subject invention also discloses a system for secure research access to aggregated hormonal health datasets, which comprises a de-identification engine configured to tokenize user identities and generalize sensitive metadata, a privacy-enforcement module implementing differential-privacy mechanisms on selected features, a cohort-construction module configured to cluster records into research-relevant groups, an analytics engine configured to compute aggregate measures including hormonal-phase distributions, cognitive-performance trends, and stress-response patterns, an application-programming interface configured to serve aggregate and summary statistics to authorized research clients, and an access-control subsystem configured to authenticate researchers, enforce usage policies, log queries, and throttle access to prevent inference attacks.

The subject invention further discloses a computer-implemented method for training hormonal-phase prediction models on hormone-labeled datasets, which comprises assembling training data comprising multimodal feature vectors containing acoustic, semantic, behavioral, and physiological attributes and associated ground-truth hormonal labels derived from clinical tests or validated cycle logs, partitioning the dataset into training, validation, and test subsets, applying preprocessing operations including normalization, dimensionality reduction, and feature selection, initializing model parameters for at least one candidate architecture selected from recurrent neural networks, transformer-based models, probabilistic graphical models, or hybrid interpretable systems, iteratively updating the model parameters to minimize a loss function measuring discrepancy between predicted and true hormonal labels on the training subset, monitoring performance on the validation subset to prevent overfitting and tune hyperparameters, evaluating the final model on the test subset to estimate generalization performance, and deploying the trained model to the inference engine of the hormonal-monitoring platform.

The subject invention also discloses a system for automated monitoring of model drift and recalibration in a hormonal-phase inference platform, which comprises a model-performance monitor configured to track prediction error metrics or proxy indicators such as consistency with user-reported cycle events over time, a drift-detection module configured to identify statistically significant degradation in model performance relative to historical baselines, a recalibration manager configured to trigger retraining or fine-tuning of the model using recent labeled or pseudo-labeled data when drift is detected, a versioning subsystem configured to manage multiple model versions and support rollback if recalibration underperforms, and a deployment controller configured to roll out updated models to clients in a staged or federated manner while logging performance and safety metrics.

The subject invention further discloses a computer-implemented method for prediction of hormone-related migraine risk using multimodal biomarkers, which comprises acquiring daily or near-daily voice samples from a user and extracting acoustic features sensitive to stress and autonomic activation, deriving semantic sentiment and stress markers from the corresponding transcripts, collecting behavioral attributes including sleep duration, perceived stress, and workload intensity, retrieving physiological indicators including heart-rate variability, skin temperature, and activity levels from wearable devices, constructing a temporally aligned multimodal feature vector for each monitoring period, inputting the feature vectors into a trained migraine-risk model that accounts for hormonal-phase context, generating a risk score representing likelihood of migraine onset within a future time window, and when the risk score exceeds a defined threshold, delivering to the user an early-warning notification together with phase-appropriate preventive interventions.

The subject invention also discloses a computer-implemented method for postpartum emotional monitoring and early-risk detection using voice biomarkers and multimodal data, which comprises inviting a postpartum user to complete brief emotional check-ins at regular intervals, capturing and analyzing the user's speech to derive acoustic indicators of prosody, vocal energy, and hesitancy, extracting semantic markers of overwhelm, sadness, or anxiety from the transcripts, incorporating behavioral data including sleep fragmentation, caregiving burden, and social support indicators, combining such features with physiological signals, if available, into unified representations over time, applying a risk-detection model trained to recognize patterns associated with postpartum depression or anxiety, computing a risk score for the user at each interval, and upon detection of sustained or high-level risk, issuing supportive interventions through the application and optionally, with user consent, furnishing a summary risk report to a clinician.

The subject invention further discloses a computer-implemented method for integrating cognitive assessment into hormonal-phase monitoring, which comprises scheduling and administering brief cognitive tasks such as reaction-time tests, working-memory challenges, and attention-switching exercises through a user interface, recording performance metrics including accuracy, latency, and variability for each task, linking each cognitive assessment to the user's contemporaneous or most recent hormonal-phase prediction and multimodal feature vector, computing residuals between observed cognitive performance and user-specific baseline expectations, identifying periods where residuals significantly deviate in association with specific hormonal phases, and storing correlations between hormonal state and cognitive-performance changes to refine both risk modeling and intervention targeting.

The subject invention also discloses a system for end-to-end orchestration of hormonal-monitoring workflows in a digital health platform, which comprises a scheduling and prompting controller configured to coordinate acquisition of voice, behavioral, physiological, and cognitive data according to user-specific schedules and preferences, a multimodal data manager configured to store, index, and retrieve feature sequences and metadata for analysis, an inference pipeline orchestrator configured to invoke sequential modules for preprocessing, feature extraction, data fusion, model inference, and post-processing, a recommendation and engagement module configured to translate inference results into personalized interventions and adaptive prompts, and a logging and observability subsystem configured to track system performance, user outcomes, and model behavior for quality assurance, compliance, and further model improvement.

The subject invention discloses a computer-implemented system for detecting hormonal fluctuations in a user, in which a mobile application executes on a personal computing device equipped with a microphone and incorporates an acoustic analysis module configured to extract speech features including pitch, jitter, shimmer, formant frequencies, spectral energy, and prosodic variation. The system further includes a semantic analysis module for identifying linguistic markers of mood, cognitive state, and stress, a behavioral data interface for receiving activity data such as sleep, exercise, diet, and stress-management behaviors, and a physiological data interface for receiving parameters such as heart rate variability, blood pressure, oxygen saturation, and menstrual cycle information. A hormonal phase prediction engine applies a trained machine learning model to these inputs to generate a predicted hormonal phase displayed to the user.

The subject invention additionally discloses a non-transitory computer-readable medium storing instructions that prompt a user to provide voice samples, capture and extract acoustic features, perform semantic analysis to detect emotional and cognitive indicators, integrate such results with behavioral and physiological data from sensors, and execute a predictive model trained to associate multimodal inputs with hormonal cycle phases, with the system presenting a predicted hormonal phase and a personalized health recommendation.

The subject invention further discloses a mobile health application for women's brain-health management including a conversational artificial intelligence interface for eliciting spoken responses at periodic intervals, a data-processing pipeline for extracting and normalizing speech features relevant to hormonal fluctuation detection, a contextual analysis engine for interpreting semantic meaning in connection with daily experiences, an adaptive recommendation system for selecting interventions based on detected hormonal states and user history, and a secure data-storage subsystem implementing privacy-preserving learning without transmitting raw audio.

The subject invention further discloses a system for detecting hormonal levels through voice biomarkers, comprising a voice-capture module, an acoustic feature extraction module, a semantic analysis module, a data-integration engine for receiving physiological and behavioral data, and a machine learning model trained to determine a hormonal phase from these combined inputs.

The subject invention even further discloses a system wherein the machine learning model may be trained using federated learning to preserve user privacy and may incorporate cognitive assessments to predict brain-fog onset, with voice biomarkers including pitch, jitter, shimmer, and formant frequency patterns.

The subject invention discloses a method for detecting hormonal phases in a user by receiving a voice sample via a microphone-equipped device, extracting acoustic features such as pitch contours, harmonic-to-noise ratio, shimmer, jitter, and spectral slope, analyzing sample content for linguistic markers correlated with hormonal changes, collecting behavioral metrics including sleep, exercise, diet, and stress levels, receiving physiological metrics including heart-rate variability, oxygen saturation, blood pressure, and menstrual cycle tracking, entering all extracted and collected signals into a trained machine-learning model, and outputting the predicted hormonal phase with a personalized lifestyle recommendation.

The subject invention additionally discloses a method for improving brain health through continuous acquisition of speech samples from voice-journaling sessions, detection of micro-variations in vocal features correlated with hormonal changes, contextualization of these variations with behavioral and physiological data, prediction of short-term cognitive and emotional risks associated with the detected hormonal state, and delivery of cognitive resilience exercises, dietary suggestions, and stress-management strategies tailored to the predicted condition.

The subject invention further discloses a method for non-invasive monitoring of hormone-related cognitive decline by establishing and storing baseline speech and health metrics for a user, detecting deviations from the baseline using daily voice-biomarker analysis, classifying deviations indicative of hormonal imbalance, triggering alerts to the user or a healthcare provider, and providing evidence-based recommendations intended to mitigate cognitive-decline risks.

The subject invention discloses a hormonal-health monitoring system that includes a voice-acquisition subsystem for prompting, recording, and transmitting user speech; an acoustic signal-processing subsystem for extracting frequency-based and temporal vocal characteristics; a semantic interpretation subsystem for detecting psychological states associated with hormonal changes; a multimodal integration engine combining voice, behavioral, and physiological data; a predictive analytics engine using a neural network trained on hormonal-cycle datasets; and an output interface for presenting detected hormonal phases and corresponding brain-health recommendations.

The subject invention additionally discloses a wearable-integrated hormonal-phase detection system including a microphone for capturing speech, a sensor suite measuring heart-rate variability, oxygen saturation, skin temperature, and motion, a local processor for performing acoustic and semantic analysis, a hormonal-fluctuation detection algorithm correlating sensor and speech data with hormonal states, and a communication module transmitting results to a paired device.

The subject invention further discloses a cloud-based hormonal-health analytics platform featuring a user interface for voice and behavioral inputs, a secure ingestion API for receiving physiological data from wearables, a distributed processing cluster for analyzing speech and semantic features, a federated machine-learning model for privacy-preserving hormonal fluctuation detection across many users, and a research portal that aggregates anonymized hormonal-phase detection data for population-level trend analysis.

The subject invention even further discloses a personalized health-recommendation system including a hormonal detection module, a knowledge base of lifestyle interventions organized by hormonal state, a rules engine selecting interventions based on user preferences and predicted hormonal phase, and a delivery subsystem providing recommendations through notifications, in-app displays, or voice feedback.

The subject invention discloses a privacy-preserving hormonal-phase detection system including a voice-analysis module that extracts acoustic features without retaining raw audio, a local inference engine trained to detect hormonal fluctuations on-device, a federated learning coordinator that updates a global model based on privacy-preserving local gradients, and an encrypted communication protocol ensuring secure data exchange while maintaining user control of personal data.

The subject invention additionally discloses a secure health-analytics system including an encryption subsystem applying differential privacy to voice-biomarker data, a tokenization engine decoupling user identity from stored data, a blockchain-based audit trail recording model updates and system access, and a user interface enabling viewing, modification, or deletion of stored records.

The subject invention further discloses a cloud-based research aggregation platform composed of numerous devices collecting anonymized voice biomarkers and behavioral data, an aggregation engine normalizing and de-identifying datasets, a machine-learning module for population-level analysis of hormonal transitions and cognitive trends, and a research access interface enabling qualified institutions to retrieve analytical summaries.

The subject invention even further discloses a system for longitudinal tracking of hormonal and cognitive health that employs recurring data-acquisition prompts for speech samples, a baseline establishment module determining normative acoustic and semantic profiles, a deviation-detection module identifying statistically significant changes, and a visualization interface presenting long-term hormonal and cognitive performance trends.

The subject invention discloses a hormonal and cognitive assessment system integrating a voice-biomarker analysis module for detecting hormonal phases, a cognitive-testing module for measuring memory, attention, and executive function, a correlation engine associating hormonal states with cognitive-assessment outcomes, and a recommendation module creating personalized interventions from combined indicators.

The subject invention additionally discloses a real-time health-alerting system including a monitoring engine analyzing ongoing or recent voice data, a detection module identifying hormonal transitions exceeding thresholds, an event-classification module determining cognitive or emotional risk, and a notification subsystem delivering alerts and recommended mitigation steps to users or clinicians.

The subject invention further discloses a digital therapeutic system for hormonal modulation and brain-health enhancement including a hormonal detection engine, a behavioral-intervention library comprising mindfulness, exercise, and stress-reduction protocols, a rules-based therapeutic scheduler choosing interventions based on the detected hormonal phase, and a feedback engine adjusting therapy based on adherence.

The subject invention even further discloses a telemedicine-compatible hormonal detection platform including a user-side application capable of performing voice-based hormonal analysis, a clinician portal displaying de-identified hormonal metrics and behavioral summaries, a secure communication bridge for transmitting clinical data, and a compliance module ensuring medical-data privacy standards.

The subject invention discloses an adaptive artificial intelligence system incorporating a user-specific hormonal-phase detection model initialized from a population model, a reinforcement learning engine updating parameters based on prediction feedback, a personalization controller weighting global and individual datasets, and an output module improving predictive precision over time.

The subject invention additionally discloses a multi-device hormonal-health management system including a first device for capturing and analyzing voice data, a second device for monitoring physiological parameters, a synchronization protocol ensuring time-aligned data exchange across devices, a centralized inference engine combining multimodal inputs to determine hormonal states, and a unified dashboard presenting hormonal, cognitive, and behavioral insights.

The subject invention further discloses embodiments in which acoustic analysis includes extraction of Mel-frequency cepstral coefficients; in which semantic analysis employs transformer-based language models; in which behavioral data includes geolocation-based activity context; and in which sequential data is analyzed using recurrent neural networks.

The subject invention even further discloses embodiments including shimmer and jitter analysis, storage of anonymized feature vectors for longitudinal monitoring, sentiment scoring for emotional-valence detection, normalization of audio amplitude before feature extraction, and predictive models employing supervised learning trained on hormone-labeled datasets.

The subject invention discloses conversational AI interfaces that dynamically adapt prompts and incorporate differential-privacy noise injection; wearable devices capable of far-field speech capture and equipped with skin-conductance sensors; contextual AI systems preserving session continuity with timestamped interactions; and text-to-speech coaching based on curated evidence-based health databases.

The subject invention additionally discloses privacy-preserving federated learning models that update during device-idle states using secure multiparty computation; longitudinal-tracking databases that produce monthly hormonal summaries and predictive forecasts; anonymized datasets generated through unsupervised clustering; and demographic-band metadata that preserves anonymity.

The subject invention further discloses embodiments in which raw audio is deleted immediately after feature extraction, intermediate feature vectors are encrypted using secure enclaves, blockchain audit logs record model updates with anonymized identifiers, rotating pseudonyms enhance privacy, and dataset normalization balances menstrual-phase distributions.

The subject invention even further discloses population-level heatmaps, deviation detection using Z-score analysis, cognitive-performance timelines, working-memory and sustained-attention tasks, phase-shifted cross-correlations linking hormones and cognition, configurable severity thresholds for alerts, recommended mitigative actions tailored to hormonal-risk states, physician-approved lifestyle protocols, and adaptive intervention difficulty based on user adherence.

The subject invention discloses clinician portals that include graphical overlays comparing hormonal predictions to symptom reports, communication bridges using asymmetric key encryption, reinforcement learning engines assigning reward values based on prediction accuracy, and systems that incorporate user-provided hormonal tracking logs for self-reinforcing adaptability.

The subject invention further discloses a method for detecting hormonal fluctuations, comprising receiving voice input from a user; extracting acoustic features; performing semantic analysis; integrating behavioral and physiological data; and predicting a hormonal phase using a trained algorithm.

The subject invention further discloses a computer-readable medium storing instructions that, when executed, cause a processor to perform the steps of the method.

The subject invention further discloses a mobile application configured to prompt voice journaling, capture audio, analyze acoustic and semantic features, integrate with wearable device data, and output hormonal phase predictions to perform the steps of the method.

The subject invention further discloses a wearable device comprising a microphone, sensor array for physiological data, and processor configured to execute the steps of method.

The subject invention further discloses a method of generating personalized health recommendations comprising detecting a hormonal phase; retrieving evidence-based interventions; and delivering tailored suggestions via a user interface.

The subject invention further discloses a method for longitudinal tracking of hormonal influences on brain health, comprising periodically repeating the steps of the method and storing trends in a secure database.

The subject invention further discloses a method for detecting perimenopausal transition through analysis of long-term voice biomarkers.

The subject invention further discloses a method for detecting mood disorders correlated with hormonal fluctuations using combined semantic and acoustic analysis.

The subject invention further discloses a method for determining cycle phase during menstruation using speech-derived biomarkers and physiological inputs.

The subject invention further discloses computer program product for implementing the method in a mobile application environment.

The subject invention further discloses a method for dynamically adapting health recommendations based on real-time hormonal detection outputs.

The subject invention further discloses a method for using anonymized hormonal phase data to generate population-level brain health risk maps.

The subject invention further discloses a conversational AI system configured to engage a user in voice interactions, capture speech features, and detect hormonal cycles.

The subject invention further discloses a cloud-based platform for aggregating anonymized hormonal detection data from multiple users for research purposes.

The subject invention further discloses a software tool integrated into telehealth systems for delivering hormonal phase reports to healthcare providers.

The subject invention further discloses a wearable patch incorporating microphones and biosensors for continuous hormonal phase detection.

In some embodiments, the subject invention further comprises applying advanced noise-robust feature-extraction techniques, wherein the acoustic analysis module additionally computes features such as spectral flux, spectral flatness, harmonic prominence, cepstral peak prominence, and glottal-closure instant estimates to enhance detection accuracy under variable ambient conditions and across different microphones and recording environments. In these embodiments, the system may dynamically select or weight specific features based on detected background noise characteristics.

In certain embodiments, the subject invention further includes a semantic-context enrichment module that augments the transcription with contextual metadata, such as topic detection, discourse segmentation, and speaker-intent inference, thereby enabling more precise detection of subtle emotional or cognitive states that correlate with hormonal fluctuations. These contextual enrichments may be used to refine the confidence score produced by the hormonal-phase inference engine.

In additional embodiments, the subject invention incorporates a behavioral-event detector configured to identify specific lifestyle events—such as skipped meals, late-night screen exposure, unusually strenuous exercise, or social withdrawal—from the behavioral and physiological logs. These detected events may be encoded as binary or graded features appended to the multimodal dataset to improve prediction resolution for hormonally influenced symptoms such as mood volatility or fatigue.

In some embodiments, the subject invention further comprises a physiological-anomaly classifier configured to detect patterns such as abrupt HRV suppression, temperature instability, or atypical cycle-phase lengths and to annotate these anomalies within the multimodal feature set. These annotations may be used to either adjust or override lower-confidence hormonal-phase predictions for improved safety and reliability.

In certain embodiments, the subject invention additionally includes a multimodal quality-index calculator that evaluates the completeness, temporal alignment coherence, and internal consistency of the aggregated voice, behavioral, and physiological data. The system may use this quality index to modulate the confidence score of the hormonal prediction or to determine whether additional user input is required before generating a final recommendation.

In additional embodiments, the subject invention further comprises fallback inference pathways that enable the system to generate hormonal-phase predictions even when one or more data streams are missing or degraded. For example, if physiological data are temporarily unavailable, the system may rely more heavily on voice biomarkers and behavioral patterns or revert to an acoustic-only or acoustic-plus-semantic inference module.

In some embodiments, the subject invention includes a temporal-attention mechanism within the machine-learning architecture, allowing the predictive model to assign increased weight to recent inputs or to historically meaningful time segments—such as luteal-phase days—thereby improving accuracy in predicting hormonally driven symptoms such as irritability, migraines, or cognitive fog.

In certain embodiments, the subject invention further comprises a phase-transition detection module that identifies not only steady-state phases but also transitional hormonal periods, such as the shift between follicular and ovulatory phases or the transition into perimenopausal irregularity. These transitions may be marked by more volatile voice biomarkers and shifts in semantic affect, which the system is trained to recognize.

In additional embodiments, the subject invention includes a model-explainability engine configured to provide users or clinicians with interpretable indicators of which features—such as increased shimmer, reduced HRV, or negative sentiment—contributed most strongly to a hormonal-phase prediction. This transparency may facilitate clinician trust and user understanding of their own hormonal patterns.

In some embodiments, the subject invention additionally incorporates a user-specific adaptation layer that fine-tunes the global model using several weeks of voice, behavioral, and physiological data unique to the user. This adaptation layer may employ lightweight fine-tuning, gating modules, or sparse parameter updates to increase personalization without requiring full model retraining.

In certain embodiments, the subject invention further includes a circadian-context subsystem that incorporates time-of-day and sleep-wake cycle information into the multimodal dataset. Because hormonal expression interacts with circadian rhythms, incorporating temporal context can refine prediction accuracy and reduce false positives associated with late-night fatigue or morning grogginess.

In additional embodiments, the subject invention includes a cross-device continuity module configured to merge data streams collected across multiple devices—such as smartphones, wearables, home speakers, or sleep-tracking sensors—into a continuous profile. The module may resolve differences in sampling rates, sensor precision, and device-specific noise characteristics to ensure seamless multimodal analysis.

In some embodiments, the subject invention further includes a hierarchical storage policy in which raw sensor data are retained only transiently, extracted features are stored longer for longitudinal modeling, and aggregated metrics are archived indefinitely for ultra-long-term trend analysis. This tiered storage approach balances privacy, model training needs, and system performance.

In certain embodiments, the subject invention also incorporates an emotional-stability predictor trained to identify moments of heightened vulnerability—such as anxiety spikes or irritability peaks—based on interactions among semantic valence, HRV suppression, and sleep-quality degradation. The system may deliver preventive interventions before emotional dysregulation fully manifests.

In additional embodiments, the subject invention further includes a real-time physiologic-voice coherence module that compares instantaneous acoustic markers with recent wearable data to detect mismatched emotional or cognitive states, such as stressed speech patterns occurring during physiologically calm periods, flagging them for potential psychological or environmental interpretation.

In some embodiments, the subject invention is configured to generate predictive forecasts of hormonal phase several days in advance by analyzing rolling trends and temporal dynamics in acoustic, behavioral, and physiological patterns. These forecasts may enable preemptive lifestyle adjustments, such as modifying sleep routines or increasing hydration before anticipated cognitive or emotional challenges.

In certain embodiments, the subject invention further includes anomaly-flagging logic capable of identifying hormonal patterns deviating from typical cycle trajectories and notifying the user that irregularities may warrant clinical attention, particularly in perimenopause, polycystic ovarian syndrome, thyroid dysregulation, or stress-induced cycle disruption.

In additional embodiments, the subject invention includes a population-level comparative model in which anonymized user data are matched to similar demographic cohorts, enabling personalized baselines for individuals who lack long-term historical data. This may improve accuracy for new users during early stages of system adoption.

In some embodiments, the subject invention further comprises a cognitive-assessment subsystem configured to administer short digital tasks measuring working memory, sustained attention, verbal fluency, reaction time, and processing speed, wherein performance metrics from the tasks are converted into numerical cognitive indicators that are appended to the multimodal feature set for hormonal-phase inference. In such embodiments, the platform may dynamically adjust task difficulty or duration based on prior cognitive performance to maintain sensitivity to subtle cognitive fluctuations linked to hormonal changes.

In certain embodiments, the subject invention includes a cognitive-fatigue detector that integrates acoustic hesitation markers, semantic complexity reductions, and slowed response behavior during cognitive tasks to identify early signs of hormone-related cognitive fog. The system may generate supportive interventions or encourage rest periods when cumulative indicators exceed predefined thresholds.

In additional embodiments, the subject invention incorporates a dual-stream assessment mode in which cognitive tasks are performed immediately before or after voice journaling, allowing the system to correlate instantaneous cognitive metrics with contemporaneous voice biomarkers and physiological measurements to improve detection of hormonally driven cognitive variability.

In some embodiments, the subject invention further integrates a telemedicine communication module enabling clinicians to review user summaries, schedule virtual consultations, and initiate secure, encrypted messaging sessions directly within the hormonal-monitoring platform. The system may automatically generate pre-visit briefing materials summarizing recent voice biomarkers, behavioral trends, and physiological anomalies to support efficient clinical evaluation.

In certain embodiments, the subject invention includes a clinician-triggered reassessment feature allowing healthcare providers to request additional voice samples, behavioral check-ins, or physiological readings when concerning trends are observed. The system may prompt the user with streamlined instructions and automatically prioritize these data streams in the subsequent hormonal-phase inference cycle.

In additional embodiments, the subject invention incorporates a remote-care escalation protocol in which severe or high-risk patterns—such as sudden cognitive decline, significant emotional dysregulation, or cycle irregularities suggestive of clinical concern—trigger automated alerts to designated clinicians or care teams, based on rules configurable by the provider.

In some embodiments, the subject invention further includes a postpartum-specific analytic layer calibrated to detect emotional shifts, fatigue patterns, and vocal-prosody reductions that are characteristic of early postpartum vulnerability. These models may account for sleep fragmentation and caregiving-induced stress to distinguish typical postpartum adjustment from potential postpartum depression or anxiety.

In certain embodiments, the subject invention incorporates infant-care context tagging, in which user responses referencing feeding cycles, nighttime awakenings, or childcare stressors are automatically annotated and correlated with voice biomarkers and physiological fatigue indicators. This allows more accurate identification of hormone-related emotional strain in the postpartum period.

In additional embodiments, the subject invention includes a maternal-support recommendation engine that prioritizes micro interventions optimized for postpartum physiology, such as hydration cues, restorative breathing exercises, emotional-regulation prompts, pelvic-floor awareness reminders, and guidance for when to seek clinical evaluation if concerning patterns persist.

In some embodiments, the subject invention integrates a perimenopause-transition classifier trained to identify cycle irregularity signatures, vocal-roughness drift, and increased cognitive-variability amplitude associated with declining ovarian hormone stability. The system may notify users when long-term patterns suggest the onset of perimenopause.

In certain embodiments, the subject invention includes a multi-month drift-analysis engine that tracks progressive changes in spectral stability, prosodic modulation, sleep fragmentation, thermoregulatory fluctuations, and HRV variability, enabling detection of early perimenopausal transition phases even in users with irregular baselines.

In additional embodiments, the subject invention further incorporates a hormone-variability severity index derived from long-range correlations among behavioral volatility, acoustic jitter patterns, mood-sentiment fluctuation, and physiological instability. The index may be used for clinical risk stratification or recommendation personalization.

In some embodiments, the subject invention includes a migraine-risk predictor trained on multimodal signatures of pre-migraine states, including increased shimmer, heightened emotional-stress semantic markers, decreased HRV, altered sleep patterns, and temperature instability. The system may deliver early-warning notifications when conditions suggest heightened migraine susceptibility.

In certain embodiments, the subject invention incorporates a trigger-context tagging module that identifies environmental or behavioral migraine triggers referenced in user speech—such as skipped meals, bright-light exposure, or chronic stress—and uses these contextual cues to refine or adjust migraine-phase risk scoring.

In additional embodiments, the subject invention includes a preventive-intervention scheduler that automatically recommends hydration routines, magnesium-rich nutritional suggestions, gentle movement, light-avoidance strategies, or preemptive relaxation protocols when the system detects escalating migraine risk related to hormonal fluctuations.

In some embodiments, the subject invention further comprises a research-mode analytics module capable of generating anonymized, population-level statistical summaries, including hormone-phase distribution curves, voice-biomarker heatmaps, and cross-correlation matrices linking behavioral patterns to physiological markers across demographic cohorts.

In certain embodiments, the subject invention includes a customizable research API that provides authenticated institutions with controlled access to de-identified multimodal datasets, enabling academic studies on the relationships between hormonal cycles, cognition, emotion, neurological risk, and long-term wellness trends.

In additional embodiments, the subject invention incorporates a synthetic-data generator capable of producing privacy-preserving artificial datasets statistically similar to real user data. These synthetic datasets may be used for model-training, benchmarking, and algorithm validation without exposing sensitive personal information.

In some embodiments, the subject invention employs a hierarchical transformer architecture in which separate transformer encoders process acoustic sequences, semantic sequences, physiological timelines, and behavioral logs independently before merging their outputs through a cross-attention fusion mechanism trained to detect multimodal hormonal signatures.

In certain embodiments, the subject invention includes a hybrid interpretable-AI architecture where a deep neural network performs the primary hormonal-phase inference while an attached rule-based expert layer enforces clinically meaningful constraints, such as preventing physiologically implausible phase transitions and refining predictions based on known cycle ranges.

In additional embodiments, the subject invention further comprises a graph-neural-network engine that models interactions among heterogeneous feature types—treating acoustic features, behavioral events, physiological parameters, and cognitive metrics as interconnected nodes—allowing the system to learn complex relational structures associated with hormonal volatility.

In some embodiments, the subject invention employs a variational autoencoder or contrastive-learning framework to learn compact latent embeddings of multimodal data, enabling improved generalization across users with diverse speech patterns, lifestyles, and physiological baselines.

### DESCRIPTION OF THE INVENTION

The present invention provides a multi-layered, AI-powered digital health platform that non-invasively detects hormonal fluctuations and their impact on brain health through analysis of voice biomarkers and multimodal user data. In exemplary operation, the platform orchestrates a coordinated series of data-collection and analysis procedures designed to capture and interpret subtle physiological and cognitive changes. The system integrates acoustic analysis of voice signals, semantic processing of spoken content, behavioral monitoring derived from user interactions, physiological sensing from wearables and other devices, and optional cognitive assessments administered through the application. These components collectively generate a comprehensive and dynamic profile of an individual's hormonal state, cognitive resilience, and emotional well-being. Unlike traditional hormonal testing methods that rely on invasive sampling of blood, urine, or laboratory-based assays at discrete time points, this invention leverages ordinary speech—produced in the course of daily life—as a continuous, non-invasive input from which physiologically meaningful indicators can be extracted. As a result, the platform enables accessible, high-frequency, real-time monitoring suitable for routine use across diverse life stages, including adolescence, reproductive years, perimenopause, and postmenopausal.

In operation, the inventive platform initiates a multimodal data-acquisition cycle by first presenting the user with an invitation to submit a voice sample, typically through a microphone-enabled device such as a smartphone, tablet, smart speaker, or wearable headset. In one sequence of use, the system displays a notification or audible prompt, the user taps or speaks a wake word to indicate readiness, and the device begins listening for active speech. Upon detecting speech activity above a threshold amplitude and duration, the platform automatically opens an audio buffer and starts capturing raw waveform samples at a predefined sampling rate, such as 16 kHz or 44.1 kHz. While recording, the system simultaneously logs contextual metadata, including timestamps, device type, microphone gain, approximate ambient noise level estimated from pre-speech segments, and device orientation if available from motion sensors. When the user finishes speaking or a maximum recording duration is reached, the platform closes the recording buffer and stores the captured audio in volatile memory for immediate preprocessing. The audio is then subjected to a sequence of preprocessing operations, including noise suppression, high-pass and low-pass filtering, and amplitude normalization, and is subsequently segmented into short analytical windows (for example, 20-40 millisecond frames with a specified overlap) suitable for acoustic evaluation.

In parallel with the audio capture process, the system queries linked modules and sensors to retrieve the most recent behavioral, physiological, and cognitive data streams, such as sleep logs, step counts, heart-rate variability records, or results of recent cognitive tasks. Once all relevant streams are retrieved, the platform performs a temporal alignment procedure that synchronizes voice-derived features with user-reported behavior and sensor-derived physiological measurements, constructing a composite dataset in which each voice sample can be interpreted in the context of concurrent or recent lifestyle and physiological conditions. This synchronized dataset is then provided as the input to a multilayer AI analysis pipeline configured to evaluate correlations between vocal biomarkers and hormonal patterns, and the output of this pipeline includes a predicted hormonal phase, an associated confidence score, and a set of individualized recommendations tailored to the user's current hormonal state, cognitive-emotional profile, and stated health goals.

The invention is a speech-analysis engine capable of evaluating both the acoustic features and semantic content of the collected voice recordings. Acoustic analysis focuses on characteristics known from clinical, physiological, or empirical studies to correlate with hormonal activity and cognitive state. These characteristics include, by way of example, mean and variability of pitch, short-term jitter and shimmer indices, overall spectral energy and its distribution across frequency bands, the positions and dynamics of vocal-tract formants, measures of prosody such as intonation contours and rhythm, harmonic-to-noise ratios, and temporal characteristics of speech such as articulation rate and duration of voiced segments. Physiologically, these acoustic parameters may be influenced by fluid retention in the tissues surrounding the vocal folds, altered muscle tone in the laryngeal and respiratory systems, variations in respiratory drive, and central neuromuscular signaling shifts associated with menstrual cycles, perimenopausal hormone fluctuations, menopausal transition, postpartum endocrine changes, or other endocrine-related conditions.

Semantic analysis complements the acoustic characterization by examining the linguistic content of the user's speech. This includes identifying emotional tone and valence, computing sentiment scores, detecting stress-related expressions, measuring the complexity of sentence structure and vocabulary, quantifying speech rate and pause distribution, and extracting contextual meaning indicative of cognitive clarity, decisiveness, or rumination. Together, the acoustic and semantic analyses provide a robust, multidimensional representation of both the user's physiological state and cognitive-emotional functioning at the time of the recording.

When processing a voice recording in a laboratory-style sequence, the speech-analysis engine first receives the raw waveform samples and subjects them to a standardized signal-conditioning protocol. In a first stage, the engine applies noise suppression algorithms, which may include spectral subtraction or adaptive filtering, to reduce background noise while preserving the fundamental characteristics of the voice. In a second stage, the engine normalizes the amplitude of the signal so that recordings made at different microphone gains or distances can be analyzed on a comparable scale. In a third stage, the engine detects and trims extended periods of silence at the beginning and end of the recording, as well as long internal gaps that exceed a defined silence threshold, in order to focus subsequent analysis on speech-active segments.

After conditioning, the engine divides the waveform into overlapping frames of fixed length and computes a suite of acoustic features for each frame. These may include fundamental frequency estimates, measurements of harmonic structure, vibratory irregularity indices such as jitter and shimmer, formant trajectories tracked over time, spectral centroid and roll-off metrics, glottal-flow indicators derived from inverse filtering, and Mel-frequency cepstral coefficients representing the overall spectral envelope. Once frame-level features are computed, the system aggregates them into session-level statistics, such as means, variances, and higher-order moments across the recording, and applies temporal smoothing to reduce the influence of transient noise events. Concurrently, the system invokes an automatic speech recognition module to convert the audio into a text transcript. The semantic-analysis component then parses this transcript to compute sentiment scores, extract emotional descriptors, estimate linguistic complexity, identify stress-related keywords and phrases, evaluate speaking pace, and quantify hesitation markers such as filler words, false starts, and prolonged pauses. Finally, the system aligns the acoustic and semantic outputs on a common timeline and performs cross-modal correlation to associate patterns of vocal quality with patterns of expressed content, resulting in a rich, multidimensional representation of the user's physiological and cognitive profile for that session.

To supplement voice-derived insights, the system incorporates behavioral data collected through multiple user-facing interaction modes. These may include guided or unguided voice journaling sessions, brief conversational check-ins at scheduled times, written or spoken daily reflections, and interactive prompts that ask the user about sleep, exercise, diet, stressors, social interactions, and overall mood. In a typical behavioral data-collection cycle, the platform first presents a prompt that is either generic or specifically tailored to the user's context. The user responds verbally or textually, and the system captures the response, timestamps it, and associates it with the relevant hormonal-phase predictions. Over time, these behavioral signals allow the platform to interpret how lifestyle factors—such as sleep quality, dietary patterns, physical activity levels, social engagement, stress-management practices, and work-life balance—interact with hormonal changes to influence cognitive performance and emotional stability.

The behavioral-monitoring subsystem operates through a methodical sequence of steps that transforms user responses into structured behavioral features. In the first step, the subsystem records the user's response to structured or open-ended prompts and stores the raw content along with metadata such as date, time, and the context of the prompt. In the second step, the subsystem processes the response to extract key information relevant to hormonal, cognitive, and emotional interpretation. For example, the subsystem may detect and quantify self-reported sleep duration, perceived sleep quality, the type and intensity of any exercise performed, dietary choices such as caffeine intake or late-night eating, subjective stress levels, self-reported fatigue, and descriptions of social or occupational events. In a third step, the subsystem converts these extracted elements into structured metadata fields—numeric hours of sleep, categorical exercise intensity levels, ordinal stress ratings, descriptive fatigue metrics, or binary variables representing presence or absence of specific behaviors. In a fourth step, each structured entry is associated with concurrent or recent physiological and voice-derived data through timestamp alignment. In a fifth step, when the user has generated multiple entries within a day or across days, the subsystem aggregates these records into a rolling behavioral profile that characterizes both short-term variability and long-term habit trends. In a sixth step, the system applies quality-control filters to detect anomalous or incomplete entries, imputes missing behavioral metrics based on historically observed patterns, and normalizes behavioral values across time so that they can be directly compared with hormonal-cycle markers on a shared scale. This stepwise integration ensures that behavioral context is consistently and accurately embedded into the hormonal prediction framework and contributes meaningfully to the refinement of personalized interventions.

Physiological metrics further strengthen the predictive accuracy of the system by providing objective measures of autonomic, metabolic, and endocrine-related activity. Wearable devices or compatible sensors may continuously or periodically record heart rate variability, resting heart rate, oxygen saturation levels, respiratory rate, electrodermal activity, skin temperature, core body-temperature estimates, step counts, energy expenditure, sleep-stage estimates, and menstrual-cycle events such as onset dates, cycle length, and self-reported symptoms. These signals help the system distinguish hormonally driven changes from those caused by non-hormonal factors such as acute illness, dehydration, environmental temperature changes, circadian disruption, or acute psychological stress. In certain embodiments, cognitive assessments administered via the application—such as reaction-time tests, working-memory tasks, symbolic-reasoning exercises, attention-switching operations, or brief problem-solving tasks—provide additional insight into the cognitive effects of hormonal transitions. These assessments enable the system to quantify changes in cognitive performance over time rather than rely solely on user self-report.

To incorporate physiological data in a systematic manner, the system periodically polls connected wearable devices or sensors at defined intervals or upon event triggers. In a typical physiological-data acquisition sequence, the platform first establishes a secure communication channel with the wearable device using Bluetooth, Wi-Fi, or an equivalent protocol. It then requests the latest available records for parameters such as heart-rate variability, resting heart rate, oxygen saturation, respiratory rate, electrodermal activity, body temperature, activity levels, and recorded menstrual-cycle events. Once the records are received, the physiological-processing module performs an initial validation step to ensure that the data are complete, correctly formatted, and associated with plausible timestamps. In a subsequent step, the module applies filtering algorithms to remove artifacts, such as motion-induced spikes in heart rate or erroneous readings produced during sensor disconnection. The module then smooths erratic readings using techniques such as moving-average filters, median filters, or adaptive smoothing algorithms. It also detects short-term deviations, including significant drops in HRV, sudden temperature spikes, irregular heart-rate patterns, or unexpected menstrual bleeding events. After cleaning and deriving features from the physiological data, the system performs a synchronization step, adjusting for differences in sampling rates, device latency, and intervals of missing data, and aligns the physiological timeline with voice-derived and behavioral features. This methodical alignment allows the AI engine to determine whether a detected acoustic or semantic anomaly is accompanied by physiological dysregulation, suggesting a global stress response or illness, or whether the anomaly is more likely to represent a hormonally driven change specific to a phase of the user's cycle.

The AI engine fuses acoustic, semantic, behavioral, physiological, and cognitive data into a multimodal analysis pipeline that can be implemented using various machine-learning architectures. In one embodiment, the engine first standardizes all input features by centering and scaling them relative to both individual baselines and population-level norms. It then constructs composite feature vectors that incorporate voice metrics (such as average jitter and prosodic variability), linguistic markers (such as sentiment scores and pause densities), behavioral attributes (such as sleep-duration indices and exercise frequency), physiological indicators (such as rolling HRV averages and cycle-stage markers), and cognitive performance metrics (such as reaction-time variability or error rates). These composite feature vectors are then passed through one or more trained models, including sequential neural networks, transformer-based models, probabilistic classifiers, or hybrid interpretable architectures capable of capturing both temporal and cross-domain relationships. The system uses these models to evaluate correlations and temporal patterns across the inputs and to generate a probabilistic prediction of the user's current hormonal phase, such as follicular, ovulatory, luteal, perimenopausal, or menopausal, along with a confidence score that reflects the quality, richness, and consistency of the underlying data.

Once acoustic, semantic, behavioral, and physiological data streams are aligned, the multimodal AI engine executes a layered analysis that proceeds conceptually in a series of inferential stages. In the first stage, the system generates a unified feature vector by concatenating or otherwise combining the standardized voice metrics, linguistic markers, behavioral attributes, and physiological indicators into a fixed-length or sequence-based representation appropriate for the chosen model type. In the second stage, the engine feeds this unified representation into a trained model that may perform tasks such as clustering the input into latent states, learning time-dependent patterns, and estimating the probability of membership in each hormonal phase. In the third stage, specialized submodules may refine the predictions by incorporating additional context, such as the user's reported cycle length or known menopausal status, or by rejecting predictions made under conditions of low signal quality. For example, a sequential model such as an LSTM may scan across several weeks of user data to examine patterns such as progressive increases in acoustic jitter combined with consistent sleep reduction as precursors to particular phases. A transformer-based model may attend selectively to combinations of features that most strongly differentiate follicular and luteal phases, such as the interaction between emotional-semantic markers and HRV instability. In the fourth stage, the system synthesizes the outputs from all internal decision layers into a single predicted hormonal phase and calculates a confidence measure derived from model stability, concordance among submodules, and completeness of the input data.

Once the hormonal state is identified, the platform provides tailored, evidence-based recommendations designed to support mental clarity, emotional balance, and cognitive resilience. In an exemplary workflow, the recommendation engine first receives the predicted hormonal phase and associated risk indicators, such as elevated stress, reduced sleep quality, or predicted cognitive vulnerability. It then queries a knowledge base of interventions indexed by hormonal phase, risk type, and user characteristics, including interventions targeting physical symptoms, mood regulation, cognitive support, and lifestyle optimization. The engine filters this intervention set based on the user's preferences (for example, preferred exercise modality), lifestyle constraints (for example, limited time availability), and recorded adherence patterns. Next, the engine ranks the candidate interventions according to predicted effectiveness, which may be derived from prior user outcomes, population-level data, or clinician contribution. The highest-ranked interventions are then transformed into personalized guidance messages presented as text notifications, voice messages, or in-app modules. Over time, as the user follows or skips recommendations and provides feedback, the system tracks adherence and perceived benefit, updating its internal models so that future recommendations increasingly align with the user's demonstrated response profile.

Upon determining the hormonal phase, the system can also execute a more detailed recommendation-generation method that follows a laboratory-style sequence. First, the system retrieves from its intervention library all records associated with the detected phase and with any co-occurring conditions inferred from the data, such as high stress or chronic insomnia. Second, the system cross-references these interventions with user-specific contraindications or preferences. For instance, the system automatically removes high-impact exercise suggestions for users with joint issues or omits caffeine-based strategies for users who report caffeine sensitivity. Third, the system estimates the expected benefit of each remaining intervention using a predictive model that considers historical outcomes and similarity to other users with comparable profiles. Fourth, the system composes a recommended plan comprising one or more prioritized interventions expressed in actionable terms. Fifth, after the plan is delivered, the system monitors behavioral, physiological, and voice-based indicators to determine whether the intervention was followed and whether it produced the desired effect on mood, sleep, or cognitive clarity. Sixth, the system uses this outcome information to adjust the future ranking and selection of interventions, thereby closing the loop and enabling long-term optimization of the therapeutic strategy.

A feature of the invention is its behavioral-design architecture, which promotes sustained engagement through carefully crafted interaction sequences that feel supportive rather than burdensome. The platform uses guided journaling prompts, conversational AI check-ins, interactive reflections, and positive-reinforcement mechanisms to encourage regular participation. On days when the user has not engaged with the system, the platform may present a short, low-effort prompt to reduce friction and reestablish engagement. On days when the user has more time, cognitive capacity, or motivation, the platform may offer deeper reflective exercises, educational content, or specialized cognitive tasks. These interactions not only enrich the data available for analysis but also foster healthier routines, improved self-awareness, and proactive self-care. Over time, as longitudinal data accumulates, the system establishes individualized baselines for acoustic features, emotional-expression patterns, sleep routines, physical activity levels, and physiological norms. The platform then uses these baselines to identify subtle deviations that may indicate emerging cognitive decline, burnout, hormonal instability, or endocrine dysregulation. When such deviations exceed dynamic thresholds, the system increases support intensity—for example, by prompting more frequent check-ins, offering targeted interventions, or suggesting clinical consultation—thereby enabling early intervention before more serious symptoms emerge.

The behavioral-design architecture operates through a methodical engagement cycle described in stepwise terms. In the first step, the platform generates a scheduled or context-triggered prompt inviting the user to complete a voice or text check-in. In the second step, the system observes whether the user responds and, if so, measures response characteristics such as latency, duration, level of detail, and affective tone. In the third step, the system scores the engagement level using these metrics and classifies the user's current engagement state along a continuum ranging from highly engaged to minimally engaged. In the fourth step, based on this classification, the system adapts the structure and frequency of future prompts, shortening and simplifying prompts when engagement is low and deepening or expanding prompts when engagement is strong. In the fifth step, the system concurrently recalibrates the user's baselines by updating statistical models that represent the user's typical acoustic range, emotional-expression patterns, sleep and activity rhythms, and physiological parameters. In the sixth step, when new data points deviate substantially from these baselines, the system flags potential hormonal or cognitive shifts and may automatically adjust the intensity of interventions, frequency of monitoring, or recommendations for clinical evaluation.

The system also supports preventive medicine by identifying early indicators of hormone-related cognitive decline and other neurological risks, and by systematically linking those indicators to concrete recommendations. When the platform detects persistent patterns of reduced speech fluency, increasing pauses, slowed articulation, or lower cognitive test performance that align with particular hormonal changes, it classifies the situation as a potential early warning for hormone-related cognitive vulnerability. In response, it may recommend targeted cognitive exercises, sleep-optimization protocols, dietary modifications emphasizing neuroprotective nutrients, stress-reduction techniques, hydration strategies, or referrals for medical consultation. These methods enable the platform to function as both a real-time monitoring tool and a long-term preventive-health assistant.

For healthcare providers, the invention serves as a diagnostic and monitoring tool that can be integrated into existing care workflows. With user authorization, clinicians may access structured reports summarizing hormonal-phase predictions, time series of voice-biomarker trends, behavioral patterns, physiological metrics, and relevant cognitive-assessment results. These reports support individualized treatment planning, facilitate medication adjustments, guide recommendations regarding hormone therapy or behavioral interventions, and support ongoing care coordination. When clinicians are involved, the system performs additional steps to generate structured, medically interpretable reports following a defined procedure. The platform first aggregates hormonal-phase predictions over a specified time interval, such as the past month, and calculates summary metrics such as phase frequency, duration, volatility, and temporal alignment with symptoms. It then generates charts and tables correlating phase patterns with voice-biomarker changes, behavioral variables, and physiological indicators. Next, the system highlights periods where the model detected elevated risk for cognitive or emotional distress. These results are organized into clinician-readable dashboards that emphasize clarity, structure, and relevance to clinical decision-making. Before transmitting any report, the system uses medical-grade encryption, verifies clinician identity and authorization, and records the transmission in an immutable audit log. After reviewing the information, the clinician may provide feedback or modify care plans, which the platform can incorporate into its recommendation engine so that future guidance reflects professional oversight.

Beyond individual monitoring, the invention also facilitates population-level research through the aggregation of anonymized, de-identified datasets. Researchers can use these data to study correlations between hormonal fluctuations, cognitive performance, emotional health, stress reactivity, sleep patterns, physiological variability, and long-term neurological risk. To support research in a controlled manner, the system uses privacy-preserving data-transformation methods before storing or sharing any aggregated datasets. In privacy-focused embodiments, the system captures audio input on the user's device, immediately performs on-device feature extraction to derive acoustic descriptors and transient semantic labels, deletes the stored audio buffer after feature computation, and packages only the resulting non-identifiable features into encrypted vectors. The system then performs hormonal-phase inference locally using stored model parameters and writes only the phase prediction and summary metrics to local storage without retaining any raw voice material. When federated learning is enabled, the device periodically computes model-weight updates based on its local data and transmits only these encrypted gradients to a central coordinator. The central coordinator aggregates updates from many devices, refines the global model, and sends updated model parameters back to devices, completing a training cycle that improves performance without exposing individual-level data.

Privacy and data security are central to all embodiments and are implemented through multiple overlapping controls. The system encrypts all collected data in transit and at rest, uses secure key-management practices, and provides user-controlled data-sharing settings that allow users to specify which data streams, if any, may be shared with clinicians or researchers. On-device processing ensures that the most sensitive audio features need not leave the device, and federated learning mechanisms enable the global model to improve using distributed training rather than centralized storage of raw user data. The modular architecture of the platform allows integration with third-party wellness applications, wearable trackers, menstrual-cycle tools, cognitive-therapy applications, and electronic health record systems. In one configuration, the platform may operate as a standalone mobile app with optional wearable integrations. In another configuration, it may run as part of a clinical ecosystem with data flowing into a hospital's electronic health record environment. In another configuration, it may integrate with consumer fitness or mental-health apps, exchanging data through secure application programming interfaces. This modular design supports deployment across use cases ranging from individual self-monitoring to full-scale clinical and research environments.

For population-level research, the system executes a structured de-identification sequence prior to making any dataset available for analysis. In the first step, each user's dataset is assigned a rotating pseudonym or token that cannot be traced back to a real identity without an access-controlled key. In the second step, all direct identifiers such as names, contact details, and device-specific identifiers are removed, and location data may be coarsened or transformed to reduce re-identification risk. In the third step, voice-related features are subjected to additional anonymization such as differential-privacy noise injection or aggregation into coarser feature bins to prevent reconstruction of individual acoustic signatures. In the fourth step, datasets with similar demographic or health characteristics may be clustered to support targeted research while preserving anonymity. In the fifth step, differences in sampling frequency, data completeness, and recording duration are normalized to ensure cross-cohort comparability. In the sixth step, the system generates aggregate analytics such as heatmaps of hormonal-phase distributions, temporal hormone-pattern graphs, and population-level correlations between hormonal states and cognitive or emotional metrics. Finally, these anonymized and aggregated datasets are made available through a secure research API that enforces authentication requirements, monitors usage patterns, and implements rate limiting to prevent misuse or excessive extraction.

As a first example of use, a user who does not manually track menstrual cycles speaks daily reflections into the application. Over several weeks, the system observes gradual increases in jitter and emotional tone that consistently precede the onset of menstruation. These recurring voice patterns, along with behavioral indicators such as decreased sleep consistency and minor fluctuations in heart-rate variability, enable the system to infer the user's luteal-phase timing. The system then predicts the user's next menstrual onset and proposes targeted interventions such as dietary adjustments to stabilize mood, hydration protocols to reduce inflammation, and sleep-protection strategies to mitigate premenstrual fatigue and irritability. These recommendations adjust dynamically based on adherence and evolving symptoms, allowing the user to receive continuous and personalized support.

In another example, a perimenopausal user experiencing intermittent brain fog records daily voice entries. The system detects increased pauses, slowed speech rate, reduced semantic complexity, and greater variability in prosody—all of which have been associated with hormonal transition states. When these voice features are correlated with disrupted sleep patterns, reduced heart-rate variability, and inconsistent thermoregulation, the system generates targeted recommendations. These may include sleep-hygiene improvements, cognitive warm-up tasks, targeted breathing exercises, lifestyle modifications to stabilize circadian rhythms, and, where appropriate, suggestions for clinical consultation if symptoms intensify or show patterns consistent with known perimenopausal transition markers.

In another exemplary scenario, a user with a history of hormonally triggered migraines records daily reflections describing stress levels and early sensory symptoms. The system identifies rising negative sentiment in the semantic content, reduced heart-rate variability in wearable-derived signals, elevated electrodermal activity, and acoustic markers of sympathetic activation such as increased shimmer, spectral instability, and tightened prosodic contours. Recognizing the user's historical pattern of pre-migraine risk states during specific hormonal phases, the platform triggers a migraine-prevention protocol. This may include hydration prompts, guided relaxation, adjustments to screen exposure, recommendations for magnesium or other evidence-supported supplements, environmental modifications, and reminders to implement clinically recommended migraine-prevention strategies. As a result, the system provides early warning and actionable steps that may reduce the frequency, duration, or severity of hormonally mediated migraine episodes.

In another example, a postpartum user completes emotional check-ins during the first months after childbirth. The system detects subtle signs of emotional strain, diminished prosody, lower speech energy, and semantic expressions of overwhelm or rumination. These voice and linguistic indicators are analyzed in conjunction with sleep fragmentation patterns, reduced heart-rate variability, and fluctuating stress markers. The platform responds with mood-support micro interventions suitable for short time windows, such as brief grounding exercises, structured breathing sessions, and low-effort self-care tasks. With the user's consent, the system also generates concise summaries for clinicians as an early screening tool for postpartum depression or anxiety, identifying shifts such as persistent reductions in emotional positivity, increased hesitation markers, or prolonged speech-latency patterns. This early-detection capability is particularly valuable given the challenges of distinguishing hormonal transitions from clinically relevant postpartum mood disorders.

The platform also supports users navigating academic, occupational, or high-performance stress in ways that align with their hormonal and cognitive patterns. For example, a college student experiencing repeated periods of late-luteal anxiety records midday reflections throughout the semester. The system quantifies cyclical increases in negative sentiment, vocal tension, and hesitation markers during specific hormonal phases and observes parallel reductions in sleep regularity and diversity of daily activities. Based on these patterns, the platform recommends cycle-aligned study schedules, structured breaks, guided breathing sessions before exams, and sleep-protection strategies during low-resilience phases. Over time, the system refines these recommendations by monitoring adherence, academic performance, and physiological responses.

In another example, a remote employee in a high-stress role uses the platform while working from home. The system detects cyclic patterns of vocal strain, reduced linguistic coherence, and elevated stress sentiment that align with specific hormonal states. These patterns, combined with indicators such as sedentary behavior and prolonged late-night device use, signal increased burnout risk. The platform responds by suggesting ergonomic adjustments, screen-time boundaries, restorative breaks, social-support interactions, and mindfulness exercises, tailoring these interventions to the user's hormonal profile and daily workload.

Additional examples illustrate further embodiments of the invention. A woman undergoing fertility treatments uses the system to track emotional fluctuations and fatigue related to different phases of her treatment cycle. The platform highlights when she is most cognitively and emotionally vulnerable and suggests allocating demanding tasks during periods of greater resilience. A professional athlete uses the platform to schedule training intensity based on hormonal-linked recovery patterns, increasing training load during phases associated with higher cognitive and physiological resilience, and emphasizing recovery and deload periods when the system detects hormonal or physiological markers of vulnerability. Another user with ADHD receives cycle-aware strategies for attention management, such as customized time-blocking templates, noise-management tools, and targeted cognitive exercises implemented during phases when attention is most likely to decline.

A teenager with irregular menstrual cycles uses the system to observe how academic performance, mood variability, and emotional volatility vary across time. The platform highlights associations between late-night device use, reduced sleep duration, and increased cognitive and emotional instability, and suggests lifestyle adjustments—such as earlier wind-down routines, reduced blue-light exposure, and stress-buffering activities—to improve both cycle regularity and daily functioning. These examples demonstrate the versatility of the invention across age groups, hormonal states, and user needs.

Across all embodiments, the invention provides a comprehensive and accessible platform for hormonal monitoring, cognitive and emotional support, clinical insight, and scientific discovery. By integrating voice biomarkers with behavioral and physiological data, applying advanced machine-learning architectures, and delivering personalized, context-aware interventions, the system enables proactive support for cognitive and emotional well-being throughout the hormonal transitions individuals experience across their lifespan. The platform's multimodal fusion approach, combined with privacy-preserving methods, clinician-integrated workflows, adaptive engagement design, and research-ready data-science infrastructure, allows it to serve simultaneously as a consumer-facing health tool, a clinical decision-support resource, and a scalable scientific research engine capable of addressing longstanding gaps in women's health and neuroendocrine research.

In further embodiments, the digital health platform incorporates advanced cognitive-assessment modules designed to capture fluctuations in attention, working memory, processing speed, and executive function that may correlate with hormonal transitions. During selected sessions or at predetermined intervals, the system invites the user to complete brief, low-burden cognitive tasks, such as reaction-time tests, n-back working-memory challenges, or pattern-recognition puzzles. These tasks are designed to take only a few seconds to a few minutes, minimizing user burden while providing meaningful cognitive markers. The platform records response times, error rates, hesitation patterns, and task-engagement metrics, and aligns these cognitive indicators with concurrent acoustic features, semantic markers, behavioral data, and physiological signals. When cognitive performance deviates significantly from the user's established baseline—such as slower reaction times during luteal-phase transitions or increased error rates during perimenopausal stages—the system integrates these findings into the hormonal-phase inference algorithm, increasing precision and enabling more tailored cognitive-support recommendations.

Cognitive-assessment workflows can also operate in stepwise laboratory-style sequences. In one such method, the platform begins by generating a prompt requesting the user to complete a designated cognitive task. Once the user initiates the task, the system measures the latency before the first response as an indicator of readiness and attentional engagement. It then records performance across multiple trials, logging correct responses, incorrect responses, and missed responses, as well as the distribution of reaction times. The system analyzes intra-task variability—such as increasing delays across trials—as potential signs of stress, fatigue, or hormonal influence on cognitive flexibility. Finally, the system standardizes the results relative to the user's historical performance and incorporates these normalized scores into the multimodal inference engine. This cognitive dimension enhances the platform's ability to detect subtle changes that may not be apparent from voice or behavioral signals alone.

In certain embodiments, the system includes telemedicine-integration features enabling clinicians to directly interact with the user through secure communication channels. The platform supports asynchronous messaging, real-time consultations, and remote monitoring of hormonal-phase predictions and associated health insights. A telemedicine-enabled workflow may begin when a clinician receives an alert indicating repeated deviations in the user's cognitive, emotional, or physiological signals. The clinician can review the system-generated report, which includes voice-biomarker trends, behavioral profiles, and cycle-phase predictions. Through the telemedicine interface, the clinician may request additional information from the user, schedule a virtual appointment, or adjust recommended strategies. The platform then incorporates clinician feedback into its intervention engine, ensuring that future recommendations reflect both automated intelligence and expert guidance. This combination of digital automation and human oversight strengthens clinical relevance and enhances patient safety.

Telemedicine interactions can also follow structured procedural steps. In one such example, the system first compiles a summary of recent hormonal-phase predictions, acoustic deviations, physiological anomalies, and behavioral trends, and encrypts the summary for secure transmission. Second, the clinician interface alerts the provider to new incoming data and displays the summary in a customizable dashboard. Third, the clinician may annotate any chart, graph, or timeline with diagnostic impressions or suggested interventions. Fourth, the system integrates these clinician annotations into the user's personalized model, refining both near-term recommendations and long-term behavioral guidance. Fifth, if the clinician determines that further evaluation is needed, the platform can automatically increase monitoring intensity, prompting the user for more frequent check-ins, cognitive tasks, or symptom logs. This closed-loop telemedicine architecture ensures continuity between remote assessments and day-to-day health monitoring.

Postpartum monitoring provides another important use case. In the months following childbirth, hormonal fluctuations can interact with sleep deprivation, emotional stress, and physical recovery in complex ways. The platform supports postpartum users by capturing daily voice samples, emotional check-ins, and cognitive indicators, and by correlating these with physiological markers such as disrupted sleep cycles and fluctuating HRV. When the system detects persistent signs of emotional strain—such as reduced prosody, increased hesitation markers, or semantic indicators of overwhelm—it generates tailored interventions aimed at supporting mood stabilization and mental clarity. These may include micro-meditations, structured breathing sessions, rest-prioritization strategies, and low-effort cognitive exercises. With consent, the system may also generate clinician-facing reports highlighting patterns consistent with postpartum depression risk, enabling early clinical engagement. This continuous monitoring is especially valuable because postpartum conditions often emerge gradually and may be overlooked during infrequent clinical visits.

In another embodiment, the system is configured for perimenopausal transition detection. Perimenopause typically involves irregular cycles, unpredictable hormone fluctuations, and shifts in cognitive and emotional stability. The invention capitalizes on its longitudinal datasets to identify early-stage perimenopausal signatures, such as prolonged luteal-phase variability, increased acoustic instability (particularly in prosody and shimmer), heightened emotional volatility, and altered sleep patterns. The system compares these signals to clinical patterns documented in population-level training datasets and generates contextual analyses explaining how the user's current trajectory aligns with known perimenopausal markers. When transition indicators accumulate, the system may present the user with informational content explaining perimenopause, encourage sleep and cognitive-support strategies, and, if warranted, recommend connecting with a clinician to explore treatment options.

Migraine prediction represents another embodiment. Hormonal migraines are frequently preceded by subtle physiological and cognitive cues that may not be recognized by the user. The platform integrates acoustic signs of laryngeal tension, reduced spectral stability, increased shimmer, and vocal stress with physiological measures such as drops in HRV, elevated electrodermal activity, and sleep-disruption signals. Semantic analysis frequently captures early warnings that the user may express indirectly, such as descriptions of irritability, pressure, light sensitivity, or stress. By combining these markers into a predictive model trained on migraine-labeled datasets, the system identifies pre-migraine states with increasing accuracy. When a risk state is detected, the platform activates an evidence-based prevention plan, which may include hydration reminders, screen-brightness reduction cues, environmental adjustments, magnesium supplementation suggestions, rest protocols, or mindfulness-based stress reduction exercises.

From a research and data-science perspective, the system's multimodal architecture creates substantial opportunities for population-level analysis of hormonal health, cognition, and emotional stability. With proper de-identification and differential-privacy methods, the collected datasets can be aggregated to analyze trends in hormonal-phase duration, menstrual irregularity patterns, correlations between voice biomarkers and cognitive outcomes, and cycle-dependent variations in sleep, stress, or emotional expression. The platform may also cluster users by demographic or health attributes to identify subgroups with similar cycle characteristics or cognitive-vulnerability profiles. Machine-learning scientists may use these anonymized datasets to refine predictive models, develop new biomarkers, or generate longitudinal heatmaps illustrating how cognitive variability tracks hormonal changes across diverse populations.

The platform may further incorporate specialized machine-learning architectures to enhance performance and interpretability. In one embodiment, a hierarchical transformer processes long-range temporal dependencies between acoustic, behavioral, and physiological features. In another, a dual-stream neural architecture separates fast-changing indicators (such as acoustic jitter) from slow-changing indicators (such as baseline HRV or sleep patterns), combining them through late-stage fusion. A probabilistic graphical model may supplement deep-learning models to provide interpretable estimates of hormonal-phase uncertainty. Additionally, an attention mechanism may identify which features are most influential in a given prediction, supporting transparency for clinicians and researchers. This modular design allows the platform to adapt to new feature types, new training datasets, and evolving scientific understanding of neuroendocrine dynamics.

Additional embodiments extend the platform's multimodal analytic capabilities by incorporating deeper contextual modeling of how environmental, behavioral, and psychosocial factors interact with hormonal fluctuations. For example, the system may analyze geolocation-derived context—such as whether the user is at home, at work, commuting, or traveling—to interpret how changes in environment influence stress expression or vocal stability. When a user regularly experiences elevated shimmer or increased negative sentiment while in a particular location, the system may infer an environmental stressor impacting emotional and hormonal equilibrium. The system may then tailor recommendations to help the user anticipate and mitigate environmental contributors to mood and cognitive variability, such as providing relaxation cues before entering known high-stress zones or suggesting structured decompression routines afterward.

In yet another embodiment, the platform incorporates time-of-day modeling to distinguish hormonal-driven changes from circadian fluctuations. The system assesses whether acoustic markers, such as increased jitter or reduced pitch stability, follow predictable circadian trends or deviate sharply in ways more typical of hormonal influence. The platform also evaluates whether cognitive performance metrics vary systematically by time of day, and whether those variations interact with cycle-phase predictions. For instance, a user might show normal circadian reductions in cognitive alertness during late evening hours but exhibit disproportionately amplified declines during perimenopausal transitions. The system integrates these temporal insights into its prediction engine, improving specificity and reducing false positives.

The invention also includes embodiments in which nutritional factors are explicitly modeled as influencing hormonal and cognitive states. The platform may prompt users to report dietary patterns, such as caffeine intake, hydration levels, or consumption of nutrient-dense foods. It may incorporate food-logging information from third-party apps or wearable-integrated sensors measuring glucose stability. When dietary patterns correlate with acoustic volatility, reduced cognitive performance, or mood deterioration during particular hormonal phases, the system can provide phase-aware nutritional recommendations. For example, the platform may suggest increasing omega-3-rich foods during high-inflammation phases or moderating caffeine intake during sensitive luteal windows to improve sleep and emotional resilience.

The system can also integrate sleep-architecture data from wearable devices that estimate sleep stages such as light, deep, and REM sleep. A specialized sequence may detect patterns such as reduced REM sleep preceding heightened emotional reactivity and correlate these patterns with rising acoustic tension or increased negative sentiment. In these embodiments, the platform not only analyzes sleep-quality metrics but also interprets how those metrics interact with hormonal patterns to influence cognitive and emotional outcomes. Users may receive tailored guidance to stabilize sleep during vulnerable phases, including bedtime routines, environmental adjustments, and stress-regulation strategies.

Other embodiments support workplace or academic performance optimization by aligning task-recommendation strategies with predicted hormonal resilience. For example, the system may identify windows in which a user's cognitive performance, emotional stability, and sleep-recovery metrics are optimal and recommend scheduling cognitively demanding tasks during these intervals. Conversely, when the system detects hormonal phases that reliably lead to reduced attention, increased distractibility, or lower mood stability, it may advise prioritizing simpler tasks, reducing cognitive load, or building additional breaks into the day. This functionality can improve productivity while reducing stress and burnout, particularly for individuals who experience significant hormonal-driven cognitive variability.

In another variation, the system integrates with digital productivity tools such as calendars, task managers, or project-management platforms. By correlating predicted hormonal phases with upcoming obligations, the platform can proactively offer phase-aware adjustments to a user's schedule. For example, if the system predicts an upcoming phase associated with emotional sensitivity and reduced sleep quality, it may recommend delaying nonessential meetings, blocking out short recovery periods, or front-loading cognitively demanding work earlier in the week. These embodiments enable harmonization of hormonal health with daily responsibilities, reducing cognitive strain and improving overall well-being.

The platform also supports specialized embodiments focused on adolescent users, who often experience irregular hormonal cycles, high academic stress, and rapid cognitive development. For adolescents, the system may emphasize low-burden journaling prompts, provide simplified educational content about hormonal health, and include behavioral strategies designed to support academic focus, emotional regulation, and sleep consistency. When the system detects patterns such as increased irritability, inconsistent sleep patterns, or declining attention markers associated with early-cycle changes, it may recommend supportive routines tailored to the adolescent's developmental stage.

In embodiments designed for menopausal and postmenopausal users, the system may shift its focus toward detecting trends indicative of long-term cognitive health risk. As estrogen levels decline, many users experience changes in verbal memory, attention, and emotional variability. The platform detects and tracks these changes over time, correlating them with sleep, stress, and physiological data, and provides cognitive-support strategies, such as memory-reinforcement exercises, structured routines, or sleep-optimization plans. Additionally, the system may generate clinician-facing summaries specifically tailored to menopausal care, supporting personalized decisions about hormone therapy, lifestyle modifications, or further evaluation.

Machine-learning architectures in the system can incorporate reinforcement-learning mechanisms that adapt to the user's responses and outcomes. In such embodiments, the system evaluates whether delivered interventions resulted in measurable improvements in sleep stability, reduced acoustic tension, improved sentiment, or increased cognitive performance. Positive outcomes reinforce the intervention-selection strategy, while neutral or negative outcomes cause the system to decrease reliance on those strategies. Over time, the platform develops a highly individualized therapeutic profile, selecting interventions that maximize benefit for each user based on their personal data patterns.

In some embodiments, the platform supports cross-device integration for users who own multiple devices, such as a smartphone, smartwatch, and smart speaker. The system synchronizes data across devices using secure protocols, ensuring that voice samples captured by a smart speaker in the home can be aligned with physiological data collected by a smartwatch and behavioral prompts viewed on a mobile device. This multi-device architecture allows passive collection of voice biomarkers in natural settings, such as during household conversations, while preserving privacy through on-device feature extraction and deletion of raw audio.

The platform may also incorporate context-aware prompting strategies. For example, if the system detects that the user is experiencing low engagement, it may make prompts shorter, less frequent, or more supportive in tone. Alternatively, when engagement is high, the platform may provide more in-depth reflective prompts or educational content. This adaptive prompting mechanism allows the system to maintain long-term engagement without overwhelming the user, ensuring sustained data collection and ongoing health benefits.

In other embodiments, the system integrates menstrual-cycle predictions with fertility-awareness methods. For users attempting to conceive or avoid pregnancy, the platform may combine voice biomarkers with physiological signals such as basal temperature variation, HRV shifts, cervical mucus logs, or ovulation test inputs to improve cycle-stage predictions. The system may then deliver fertility-related guidance, such as identifying suspected ovulatory windows or providing cycle-regularity insights, while maintaining privacy through encrypted storage and user-controlled data-sharing settings.

In further embodiments, the platform incorporates environmental-sensor data such as temperature, humidity, light exposure, and air quality. These measurements, gathered from wearable devices or smart home systems, can influence sleep, mood, and physiological stress responses. The system correlates these environmental factors with vocal and physiological biomarkers to determine whether external conditions may be amplifying hormonal vulnerability. For example, high temperatures and poor air quality may worsen sleep and emotional stability during particular phases. The platform uses these correlations to recommend environmental modifications, such as adjusting indoor temperature, increasing hydration, or optimizing lighting patterns.

Finally, additional embodiments support integration with clinical laboratory data when available. Users undergoing lab-based hormonal testing—for example, estradiol, progesterone, LH, FSH, thyroid hormones, or cortisol—may manually input or sync these results with the platform. This allows the AI model to recalibrate itself using high-quality ground-truth hormonal measurements, improving future inference accuracy. In research contexts, such integrated datasets may be used to enhance model performance, validate voice biomarkers against gold-standard hormonal tests, and discover new relationships between speech, physiology, and endocrine function.

Additional embodiments of the platform support nuanced modeling of psychosocial context by incorporating data from user-reported interpersonal interactions, relationship stressors, and social support patterns. For example, when the semantic-analysis module detects recurrent linguistic indicators of interpersonal strain—such as increased references to conflict, overwhelm, or social withdrawal—the system evaluates whether these patterns coincide with specific hormonal phases or emerge independently as psychosocial stressors. When combined with physiological markers such as reduced HRV or increased electrodermal activity, the platform can distinguish hormonally mediated emotional sensitivity from prolonged external stress, thereby refining both hormonal-phase predictions and recommendation strategies. The system may then recommend context-sensitive interventions such as communication frameworks, psychosocial coping strategies, boundary-setting guidance, or prompts encouraging supportive social engagement.

In another set of embodiments, the platform incorporates audio-context detection, enabling it to determine whether the user is speaking in a calm environment, a noisy workplace, a quiet room, or outdoors. The system evaluates how acoustic context influences vocal biomarkers, adjusting confidence levels and analytical weight accordingly. For example, elevated jitter or shimmer detected during a crowded commute may receive less interpretive weight than similar markers detected in quiet settings. By iteratively modeling context, the platform can more accurately isolate physiologically driven vocal changes from environmentally induced artifacts. This context-modulated analysis improves resilience of the system across real-world conditions, ensuring high-fidelity insight even when users record voice samples in highly variable environments.

The platform may also incorporate advanced fatigue modeling by integrating multiple user signals such as reduced speech rate, increased hesitation markers, lower cognitive-task performance, reduced step counts, and decreased sleep efficiency. When fatigue markers consistently appear during particular hormonal phases, the system provides fatigue-mitigation strategies such as strategic napping, hydration reminders, circadian-aligned light exposure, or microbreak scheduling. In occupational settings, this functionality helps users maintain productivity and emotional regulation by identifying windows of vulnerability and recommending compensatory adjustments to work routines, screen time, or task prioritization.

For users with chronic conditions that interact with hormonal fluctuations—such as PCOS, endometriosis, thyroid disorders, or autoimmune conditions—the platform can incorporate condition-specific modeling. For example, users with PCOS often experience irregular cycles and metabolic instability. In such cases, the system relies more heavily on behavioral, semantic, and physiological indicators, such as HRV instability, sleep fragmentation, and mood variability, to infer probable hormonal patterns. Over time, the platform builds individualized hormonal signatures that do not depend strictly on conventional cycle timing, enabling more accurate predictions even for users whose endocrine rhythms diverge from typical patterns.

Additional embodiments enable the platform to function as a resilience-tracking system during major life transitions, such as pregnancy, postpartum recovery, menopause, career changes, caregiving responsibilities, or acute stress periods. By monitoring shifts in voice biomarkers, behavioral routines, and physiological responses, the system identifies when the user's resilience is declining or when cumulative stress is exceeding adaptive thresholds. In such cases, the platform recommends stabilization strategies tailored to the user's life context, such as structured rest routines, mental-load reduction strategies, cognitive pacing, social-support cues, or environmental adjustments. When appropriate and authorized, the system can also notify a clinician or caregiver to facilitate early intervention.

In other embodiments, the platform incorporates meta-learning techniques to identify emerging patterns across large populations without compromising individual privacy. Through federated analytics, the system can discover previously unknown correlations—such as associations between specific vocal features and early perimenopausal transition, or links between sleep-fragmentation signatures and mid-cycle mood instability. These insights improve predictive capabilities of the global model while expanding scientific knowledge about women's brain health and hormonal effects. Researchers may use aggregated trend analyses, heatmaps, and temporal graphs to explore cohort-level relationships between hormonal states and cognitive metrics, identify high-risk phenotypes, or evaluate the impact of lifestyle interventions across demographic groups.

The platform may also support adaptive educational modules that deliver content about hormonal health, cognitive resilience, stress physiology, sleep science, and emotional well-being. When the system detects user curiosity—such as when the user asks questions or expresses confusion in journaling entries—it provides customized explanations that connect the user's real-time biomarkers with understandable physiological mechanisms. For example, if a user exhibits rising vocal tension and sleep reduction, the system may provide a short educational segment explaining how progesterone fluctuations can increase sensitivity to stress during certain phases, followed by practical steps to improve coping. These modules enhance user understanding, increase adherence to recommendations, and promote long-term health literacy.

Further embodiments support integration with mental-health screening protocols to detect patterns consistent with mood disorders, anxiety disorders, ADHD, or early cognitive decline. When the system detects semantic markers of hopelessness, elevated negative sentiment, or cognitive disorganization, particularly when combined with physiological or sleep disruptions, it can offer early supportive interventions and, with consent, notify a clinician for further evaluation. In postpartum use cases, for instance, the system may detect early linguistic markers of postpartum depression, such as decreased emotional range, increased descriptors of overwhelm, or reduced prosody. It can then initiate an escalated monitoring protocol and provide supportive micro-interventions tailored to postpartum emotional needs.

Additional embodiments incorporate multi-hormone modeling, enabling the system to distinguish speech patterns associated with estrogen decline from those associated with progesterone peaks or cortisol-driven stress responses. By integrating cortisol-proxy signals such as HRV reduction and increased sympathetic arousal indicators, the system can determine when vocal markers reflect endocrine transitions rather than psychological stress alone. This multi-hormone modeling enables the platform to differentiate luteal-phase tension from chronic stress responses, menopause-related instability from sleep debt, and postpartum endocrine shifts from generalized mood disorders.

The platform can also function as a predictive calendar system, automatically forecasting future hormonal phases and their likely cognitive or emotional effects. For instance, based on trends in the user's voice biomarkers, behavioral metrics, and physiological deviations, the system may predict reduced verbal fluency or increased emotional sensitivity several days in advance. This allows users to proactively plan for vulnerable windows, schedule rest periods, adjust expectations, or implement coping strategies ahead of time. For athletes, the predictive calendar might inform optimal training cycles, while for students, it might guide study planning and exam preparation.

In certain embodiments, the platform also detects “phase drift,” in which hormonal cycles gradually shift earlier or later over time. This feature is particularly helpful for users with irregular cycles or perimenopausal variability. The platform detects drift by comparing current acoustic and physiological signatures with historical baselines and identifying gradual temporal misalignment between predicted and observed biomarkers. It then recalibrates cycle predictions and informs the user of shifting patterns, providing guidance on lifestyle modifications that may help stabilize cycle timing or mitigate cognitive-emotional impacts.

Finally, expanded embodiments include community-facing features, allowing users—if they choose—to anonymously compare their patterns with aggregated, anonymized population insights. For example, a user might learn that individuals with similar HRV trajectories and sleep disruptions often experience cognitive dips during the same phase, or that specific interventions (such as magnesium supplementation or structured breathing routines) show higher effectiveness for users within their demographic group. These community-informed insights empower users to contextualize their experience while maintaining total privacy.

In another group of embodiments, the platform incorporates predictive modeling for sleep-cycle interactions with hormonal states. Because hormonal fluctuations exert measurable influence on circadian regulation, REM latency, and sleep efficiency, the system examines patterns such as increased nighttime awakenings, decreased deep-sleep intervals, and changes in sleep-onset latency. When these patterns co-occur with acoustic or semantic markers of hormonal transitions—for example, increased shimmer during early morning recordings or decreased speech-rate consistency after poor sleep—the platform identifies the interaction as a compounded risk factor. It then delivers sleep-specific interventions, such as relaxing pre-bed routines, circadian-aligned light exposure strategies, and recommendations for structured wake times to stabilize circadian rhythm during hormonally vulnerable phases. In some embodiments, the system can also estimate the likelihood of “sleep-fragmentation cascades,” in which hormonal fluctuations amplify sleep deficits across several days, leading to predictable declines in cognitive clarity or emotional resilience.

Additional embodiments incorporate nutritional-pattern modeling that analyzes self-reported diet logs, spoken dietary reflections, and physiological correlates such as glycemic variability inferred from wearable proxies. The platform examines how dietary patterns interact with hormonal states—for example, identifying when luteal-phase cravings for carbohydrate-rich foods correlate with acoustic markers of fatigue or mood instability. The system may recommend targeted nutritional interventions such as anti-inflammatory meal choices, phase-specific macronutrient balancing, hydration strategies, and micronutrient supplementation. When combined with semantic markers indicating reduced appetite, emotional eating, or dietary inconsistency, the system refines nutritional recommendations and monitors adherence.

Embodiments tailored to users with high cognitive demands—such as students, executives, healthcare workers, and caregivers—incorporate advanced cognitive-load modeling. The platform analyzes reaction-time tasks, working-memory performance, error rates, speaking coherence, and fluctuation in linguistic complexity to detect periods of cognitive overload that align with hormonal phases. When cognitive load reaches thresholds associated with reduced executive function or heightened emotional reactivity, the system recommends cognitive pacing, structured task sequencing, microbreaks, or modified workflows designed to prevent burnout. The system may also provide anticipatory support by predicting future cognitive-load vulnerability based on historical patterns and upcoming hormonal phases.

Another group of embodiments includes specialized support for menstrual irregularity, including adolescents establishing early cycles, users with endometriosis, users with PCOS, and individuals with unpredictable hormonal fluctuations. For these groups, the system shifts emphasis away from fixed cycle-day models and toward dynamic inference driven by real-time voice biomarkers, sleep metrics, HRV variability, and semantic stress indicators. When the system detects irregularity across several cycles, it re-calibrates prediction models to rely more heavily on hormone-correlated voice features such as pitch instability, prosodic flattening, or increased jitter. The platform may also deliver educational modules explaining cycle irregularity and recommending lifestyle changes or clinical follow-up when appropriate.

In further embodiments, the platform incorporates environmental-context modeling to assess how heat, humidity, altitude, or air quality influence physiological markers and interact with hormonal states. For example, when a user records voice samples at high altitude and the system detects lower oxygen saturation and altered respiratory patterns, it adjusts acoustic weighting to avoid false attribution of vocal instability to hormonal changes. Similarly, if environmental stressors such as heatwaves correlate with increased heart rate or reduced sleep efficiency, the system contextualizes these effects and adapts its recommendations to reduce environmental load, such as through hydration strategies or temperature regulation techniques.

Additional modeling embodiments evaluate long-term emotional trajectories using semantic markers of optimism, resilience, worry, and rumination. When the system detects declining emotional resilience across multiple hormonal cycles—such as increasing references to overwhelm, reduced linguistic positivity, and lower prosodic variability—it may classify the user as entering an extended psychosocial vulnerability window. In response, the system increases the frequency of supportive prompts, recommends targeted emotional-regulation exercises, or—if authorized—alerts a clinician for early intervention. This long-term resilience tracking helps prevent escalation into chronic mood disorders by identifying downward trends early.

Another set of embodiments integrates relational and family-context modeling. For users who speak about caregiving responsibilities, parenting challenges, or relationship stress, the semantic-analysis engine identifies these themes and examines their interaction with hormonal phases. For example, if a postpartum user repeatedly references overwhelm or sleep deprivation, the system identifies these as amplifiers of hormonal vulnerability and recommends tailored support strategies such as time-based self-care rituals, micro-rest strategies, or prompts encouraging users to request social or partner support. Similarly, when a perimenopausal user describes challenges with aging parents or workplace dynamics, the platform contextualizes these additional stressors when generating recommendations.

In certain embodiments, the platform incorporates enhanced migraine-prediction modeling, expanding on core migraine-detection functionality. The system combines acoustic signatures associated with sympathetic activation—such as increased shimmer, reduced spectral stability, and tension in upper formant ranges—with physiological markers such as reduced HRV, increased resting heart rate, elevated skin temperature, and sleep disruption. When these features align with historically migraine-prone hormonal phases, the system triggers a pre-migraine alert. It may also offer preventive strategies such as hydration targets, magnesium supplementation prompts, light-reduction interventions, or recommendations to avoid high-strain cognitive tasks during the predicted high-risk window. Over time, the system learns user-specific migraine signatures and fine-tunes predictions for greater precision.

Embodiments supporting perimenopausal transition detection include long-term trend analysis of acoustic irregularity, prosodic flattening, semantic markers of cognitive fog, increased sleep fragmentation, and HRV instability. The platform evaluates whether these patterns show increasing volatility across months—a hallmark of perimenopausal transition. When detected, the system informs the user of possible perimenopausal indicators, provides tailored coping strategies, and recommends clinical consultation if symptoms significantly affect functioning. These embodiments help individuals navigate the perimenopausal transition more smoothly by identifying early, subtle changes often missed in routine clinical encounters.

Additional embodiments leverage data-science functions for cluster discovery, anomaly detection, and predictive risk scoring. For example, the system may identify subtypes of users whose hormonal transitions manifest primarily through emotional volatility versus those whose primary indicators are cognitive or physiological. This clustering supports personalized model refinement and improves clinical relevance. Anomaly detection modules identify when a particular data point—such as an abrupt HRV drop or unusually flat prosody—falls outside user-specific norms, prompting timely intervention. Population-level risk scoring provides insight into whether particular users exhibit patterns similar to cohorts at elevated risk for depression, cognitive decline, or burnout.

In machine-learning-focused embodiments, the platform employs specialized architectures such as multimodal attention transformers, hierarchical recurrent networks, graph-based temporal models, or Bayesian neural networks designed to assess uncertainty. These architectures enhance the platform's ability to parse long-range dependencies, identify interactions between heterogeneous data streams, and quantify confidence in predictions. In some configurations, the platform uses interpretable AI techniques—such as SHAP values or attention maps—to display, when clinically appropriate, which features contributed most strongly to a prediction. These interpretable outputs support clinician trust and regulatory transparency.

Finally, the platform may include predictive modeling for hormone-therapy response monitoring. For users undergoing hormone therapy—such as menopausal hormone therapy, fertility treatments, or gender-affirming hormone therapy—the system tracks changes in vocal biomarkers, physiological measures, and cognitive metrics over time. It identifies whether therapy is producing stabilization, improved cognitive clarity, or reduced emotional volatility. When therapy-related side effects emerge—such as increased irritability, sleep disturbance, or unexpected vocal changes—the system may flag these patterns and recommend clinician review. This continuous monitoring supports safer, more customized hormone-therapy management.

In further embodiments, the system incorporates contextual activity modeling to evaluate how a user's daily routines amplify or buffer hormonal influences on cognitive or emotional functioning. For example, if a user consistently records voice entries immediately after waking, the system compares early-morning acoustic stability and semantic clarity across hormonal phases to detect hormonal effects on sleep inertia and cognitive readiness. Conversely, if a user submits most recordings during commuting hours, the platform adjusts for environmental noise and stress factors associated with transit, ensuring the model does not over-attribute stress-induced vocal strain to hormonal fluctuations. By using contextual weighting, the system preserves inference accuracy across diverse recording conditions and lifestyle patterns.

Embodiments of the system may additionally incorporate workload-forecasting models that anticipate cognitive strain based on a user's scheduled activities, task load, or anticipated stress events. For instance, when the platform detects upcoming high-exertion periods—such as exams, work deadlines, caregiving intensification, travel, or emotionally heavy events—it cross-references these with predicted hormonal phases to estimate compound risk. If upcoming stressors coincide with historically vulnerable hormonal windows, the system proactively recommends workload adjustments, advanced rest strategies, or emotional-buffering exercises. This anticipatory modeling supports users engaged in high-performance environments and prevents performance declines or emotional dysregulation before they occur.

Another group of embodiments enhances the modeling of emotional microexpression through fine-grained semantic and acoustic markers. The system detects subtle linguistic shifts such as increased use of first-person singular pronouns, negative intensifiers, or cognitive-processing verbs indicative of rumination or overwhelm. Acoustically, the system identifies microvariations such as breathiness, glottal tension, and cyclic patterns of fundamental frequency instability. When combined, these features allow the system to detect emerging emotional difficulty—such as irritability, sadness, or anxiety—even when users do not explicitly state such feelings. This allows earlier and more precise delivery of mood-support interventions timed to hormonal and behavioral context.

Embodiments relating to adolescent hormonal-health monitoring place additional emphasis on cycle establishment, emotional regulation, and academic functioning. Since adolescents may experience highly irregular early cycles, the system relies heavily on voice biomarkers and behavioral trends rather than fixed calendar intervals. When the platform identifies heightened emotional reactivity, reduced sleep consistency, and variability in academic productivity aligned with early-cycle transitions, it recommends stabilization routines such as structured bedtime schedules, reduced stimulants, hydration strategies, and simplified task planning. Educational modules may also be presented to help adolescents understand the relationship between hormonal changes and cognitive-emotional variability during development.

In embodiments designed for postpartum users, the system includes specialized detection modules tuned to postpartum-specific endocrine patterns such as rapid estradiol withdrawal, disrupted sleep architecture, lactation-related hormonal cycles, and emotional fluctuations typical of the early months after childbirth. The platform analyzes acoustic indicators of exhaustion—such as reduced prosody and slower articulation—alongside semantic cues reflecting overwhelm, loss of identity, or adjustments to caregiving workload. The system then recommends micro-rest strategies, emotional-regulation exercises, hydration reminders, simple self-care tasks, and social-connection prompts suited to the tight time constraints of early parenthood. When risk indicators such as flat affect, marked reduction in vocal variability, or increasingly negative semantic themes persist, the system may—upon the user's authorization—generate clinician summaries for postpartum depression screening.

Additional embodiments address the needs of users experiencing premenstrual dysphoric disorder (PMDD) or severe PMS. The platform examines cyclical patterns of semantic negativity, emotional intensity, and prosodic flattening during the late luteal phase and correlates them with physiological data such as reduced HRV and temperature irregularities. When a PMDD-like pattern is detected, the system provides phase-timed interventions such as cognitive defusion exercises, symptom journaling, anti-inflammatory nutritional guidance, and preemptive sleep stabilization strategies. If symptoms escalate, the platform may recommend clinical consultation to consider additional medical or psychological support.

In support of individuals undergoing fertility treatments, the system provides high-frequency monitoring of emotional, cognitive, and physiological signals that correspond to treatment phases such as stimulation, trigger, retrieval, implantation, and two-week wait intervals. The platform detects variability in emotional tone, fatigue markers, and cognitive clarity that commonly accompany fertility medications. When deviations exceed user-specific thresholds, the system recommends energy-conservation strategies, mood-regulation techniques, or support prompts encouraging social connection during emotionally challenging windows. Over multiple treatment cycles, the platform identifies personalized patterns of vulnerability and resilience, allowing for increasingly precise and supportive guidance.

Embodiments designed for athletic populations incorporate performance-aligned hormonal modeling. For endurance athletes, strength athletes, or individuals engaged in high-intensity training, the system evaluates how hormonal phases impact recovery, stamina, neuromuscular control, and mental focus. For example, during low-HRV, high-fatigue luteal-phase windows, the platform may recommend recovery-oriented training, reduced intensity, or increased sleep duration. Conversely, during follicular phases associated with higher resilience and improved neuromuscular activation, the system encourages increased load, interval training, or skill acquisition tasks. This cyclical periodization improves long-term performance and decreases injury risk through hormone-aware training adaptations.

Embodiments tailored to individuals with ADHD or executive-function vulnerabilities emphasize attention stabilization strategies aligned with hormonal variability. The platform monitors linguistic markers of distractibility, such as increased filler-word usage, semantic drift, and slower task-related articulation. Acoustic markers such as inconsistent pacing or abrupt shifts in prosody may also signal attention instability. When these features align with specific hormonal phases, the system recommends structured time blocks, minimized multitasking, noise-reduction environments, and short cognitive warm-up tasks to stabilize attention.

In another group of embodiments, the platform incorporates relational-social modeling, analyzing how social engagement patterns shift across hormonal phases. The system evaluates semantic references to conflict, emotional withdrawal, irritability, or social fatigue and correlates these with hormonal indicators. When users are at heightened risk for interpersonal conflict or social misinterpretation, the system recommends strategies such as communication pacing, quiet downtime, or intentional empathy exercises. This fosters improved relationship stability and reduced conflict triggered by hormone-related emotional vulnerability.

Research-oriented embodiments leverage the platform's anonymized datasets to advance understanding of hormonal effects on cognition, emotion, and physiology at scale. These embodiments include clustering algorithms that identify population-wide subtypes—such as users whose hormonal shifts predominantly affect sleep vs. those with strong emotional or cognitive responses. Researchers may also analyze multimodal correlations, such as whether decreased spectral stability in voice corresponds to emotional volatility in particular demographic bands. Heatmaps, longitudinal phase-transition graphs, and cohort-wide cognitive trendlines support scientific discovery and help refine medical understanding of hormonal-brain interactions.

In advanced machine-learning embodiments, the platform implements architectures tailored to multimodal temporal data, such as multimodal transformers with cross-attention layers, variational sequence models capable of handling missing data, and event-based temporal convolutional networks tuned to irregular sampling intervals. These architectures increase robustness to real-world variability in user behavior, device usage, and recording conditions. In some embodiments, Bayesian neural networks estimate model uncertainty, enabling the system to identify low-confidence predictions and request additional input. Interpretability modules may provide emphasis maps or weighted-feature visualizations to clinicians, enhancing trust and regulatory compliance.

Embodiments supporting clinical integration allow seamless data sharing with healthcare providers through encrypted APIs and clinician dashboards. The platform organizes multimodal data into interpretable summaries containing predicted hormonal phases, cognitive and emotional trajectory graphs, risk alerts, and cycle trends. Before clinician transmission, all data undergoes encryption, de-identification, and access-validation procedures. Clinicians may annotate findings, adjust care recommendations, or flag concerning trends, with these professional inputs feeding back into the adaptive recommendation engine. This integration creates a feedback loop between user-generated data, automated inference, and expert oversight.

In still further embodiments, the platform incorporates predictive modeling for burnout and chronic stress. Trends such as declining HRV, chronic late-night device use, sustained negative semantic content, and long-term declines in speech fluency can signal that hormonal transitions are compounding systemic stress. The platform responds with burnout-prevention strategies such as structured rest periods, workload triaging, or emotional-support exercises. If risk remains high despite interventions, the system may recommend clinical evaluation.

Across all these embodiments, the system continuously learns, adapts, and recalibrates to the user's evolving hormonal, cognitive, behavioral, and emotional landscape. By capturing subtle biomarkers embedded in daily speech, contextualizing them with behavioral and physiological data, and applying advanced machine-learning analysis, the platform provides a personalized, non-invasive window into brain-hormone interactions. This enables early intervention, improved self-understanding, enhanced cognitive and emotional stability, and meaningful clinical insights throughout the user's lifespan.

1. A computer-implemented system for detecting hormonal fluctuations in a user, the system comprising:
a mobile application executable on a personal computing device having a microphone;
an acoustic analysis module configured to process audio input to extract speech features including pitch, jitter, shimmer, formant frequencies, spectral energy, and prosodic variation;
a semantic analysis module configured to identify linguistic markers of mood, cognitive state, and stress from said audio input;
a behavioral data interface configured to receive data indicative of user activity including sleep, exercise, dietary intake, and stress management practices;
a physiological data interface configured to receive data from wearable devices including heart rate variability, blood pressure, oxygen saturation, and menstrual cycle information; and
a hormonal phase prediction engine configured to apply a trained machine learning model to said speech features, linguistic markers, behavioral data, and physiological data to output a predicted hormonal phase to the user interface of the application.

2. The system of claim 1, wherein the acoustic analysis module further extracts Mel-frequency cepstral coefficients (MFCCs).

3. The system of claim 1, wherein the semantic analysis module employs transformer-based natural language processing.

4. The system of claim 1, wherein the behavioral data interface is further configured to receive geolocation-based activity context.

5. The system of claim 1, wherein the hormonal phase prediction engine applies a recurrent neural network trained on sequential user data.

6. A non-transitory computer-readable medium storing instructions that, when executed by one or more processors, cause the processors to:
prompt a user to provide a voice sample via a guided or unguided voice journaling interface;
capture the voice sample and extract acoustic features including time-domain and frequency-domain parameters;
perform semantic analysis of the voice sample to detect stress indicators, emotional states, and cognitive clarity;
Integrate said acoustic and semantic results with behavioral data acquired from user input and physiological data from connected sensors;
execute a predictive model trained to associate multimodal inputs with hormonal cycle phases; and
display an indication of the detected hormonal phase and a corresponding personalized health recommendation.

7. The method of claim 6, wherein extracting acoustic features includes computing shimmer percentage and jitter percentage.

8. The method of claim 6, further comprising storing anonymized feature vectors for longitudinal comparison.

9. The method of claim 6, wherein semantic analysis is performed using sentiment scoring to detect emotional valence.

10. A mobile health application for women's brain health management, the application comprising:
a conversational AI interface configured to elicit spoken responses from a user at periodic intervals;
a data processing pipeline for extracting and normalizing speech features relevant to hormonal fluctuation detection;
a contextual analysis engine for interpreting semantic meaning in relation to user-reported daily experiences;
an adaptive recommendation system for selecting interventions based on detected hormonal states and historical user data; and
a secure data storage subsystem implementing privacy-preserving learning techniques to train predictive models without transmitting raw audio to external servers.

11. The non-transitory computer-readable medium of claim 1, wherein the instructions further cause the processor to normalize audio amplitude prior to feature extraction.

12. The non-transitory computer-readable medium of claim 10, wherein the predictive model comprises a supervised learning classifier trained on hormone-labeled datasets.
